(19) United States
(12) Patent Application Publication
(10) Pub. No.: US 2026/0051368 A1
(43) Pub. Date: Feb. 19, 2026

(54) GUARDRAILS FOR CONDITIONAL MOLECULAR GENERATION

(71) Applicant: NVIDIA Corporation, Santa Clara, CA (US)

(72) Inventors: Maria KORSHUNOVA, Sunnyvale, CA (US); Yuxing PENG, Santa Clara, CA (US); Michelle Lynn GILL, New York, NY (US); Emine KUCUKBENLI, Melrose, MA (US); Yonatan ISRAELI, Los Altos, CA (US); Guoqing ZHOU, Placentia, CA (US); Timur RVACHOV, Brooklyn, NY (US)

(21) Appl. No.: 18/807,808

(22) Filed: Aug. 16, 2024

(51) Int. Cl. G16C 20/30; G16C 20/50; G16C 20/70

(52) U.S. Cl. CPC G16C 20/30; G16C 20/50; G16C 20/70

(57) ABSTRACT

In various examples, a technique for providing a guardrail for conditional molecular generation includes inputting a latent representation of a molecule generated using a trained generative model during a first generative instance into one or more classifiers. The technique also includes generating, via execution of the classifier(s) based on the latent representation, one or more scores, wherein each score represents a predicted measure of a different undesired attribute for the latent representation. The technique further includes determining that the score(s) are not within one or more acceptable ranges and in response to the determination, preventing the trained generative model from generating one or more second latent representations of the molecule, wherein the preventing includes causing the trained generative model to generate, based at least on the score(s), one or more third latent representations associated with the molecule over one or more additional generative instances following the first generative instance.

### TECHNICAL FIELD

Embodiments of the present disclosure relate generally to machine learning and generative models, and more specifically to guardrails and other model alignment techniques for conditional molecular generation.

### BACKGROUND

Generative models are increasingly used during drug discovery to generate a diverse range of novel molecular structures that can potentially serve as drug candidates. For example, a deep learning model may be used to generate hundreds to thousands of molecular structures that are predicted to have a set of desired properties. The generated molecular structures may then be evaluated as potential drug candidates based on various criteria, such as (but not limited to) predicted binding affinity to a target protein, binding selectivity of the molecular structures, efficacy, bioavailability, hydrophobicity, and/or whether or not the desired properties are found in the molecular structures. Consequently, generative models can accelerate the discovery of novel molecules over conventional molecular design techniques that involve the use of human expertise to propose, synthesize, and test new molecules.

However, generative models are also capable of generating molecules that are toxic, harmful, and/or otherwise undesirable. For example, a deep learning model that is trained on a dataset of known molecules may inadvertently synthesize potentially toxic molecules that share structural similarities with known toxins or harmful substances. In another example, a generative model may be intentionally (e.g., by a bad actor) prompted, guided, and/or otherwise used to generate biochemical weapons, narcotics, toxins, and/or other substances with harmful properties.

Existing approaches for detecting toxic, harmful, and/or undesirable molecules involve the use of experimental techniques, such as cellular and tissue assays, to determine the effects of individual molecules on cells and/or other biological systems. However, these experimental techniques are time consuming and resource-intensive and cannot be scaled to the large number of molecular structures that can be generated by a generative model.

Other approaches for evaluating toxicity and/or other properties of molecules include Quantitative Structure-Activity Relationship (QSAR) models that relate the structures and/or properties of molecules to toxicity, biological activity, and/or other types of responses and/or outcomes. However, QSAR models tend to have limited predictive power and generalize poorly to new molecular structures.

As the foregoing illustrates, what is needed in the art are more effective techniques for detecting and/or managing the generation of toxic and/or otherwise undesirable molecules.

### BRIEF DESCRIPTION OF DRAWINGS

FIG. 1 illustrates a block diagram of a computing system configured to implement one or more aspects of at least one embodiment;

FIG. 2 is a more detailed illustration of the simulation engine, training engine, and execution engine of FIG. 1, according to at least one embodiment;

FIG. 3A illustrates how the machine learning models of FIG. 1 are used to provide inference-time guardrails for molecular generation, according to at least one embodiment;

FIG. 3B illustrates how the machine learning models of FIG. 1 are used to provide guardrails for conditional molecular generation, according to at least one embodiment;

FIG. 3C illustrates how the machine learning models of FIG. 1 are used to provide a training-time guardrails for molecular generation, according to at least one embodiment;

FIG. 3D illustrates how the machine learning models of FIG. 1 are used to provide guardrails for instruction-tuned molecular generation, according to at least one embodiment;

FIG. 4 illustrates a flow diagram of a method for providing an inference-time guardrail for molecular generation, according to at least one embodiment;

FIG. 5 illustrates a flow diagram of a method for providing a guardrail for conditional molecular generation, according to at least one embodiment;

FIG. 6 illustrates a flow diagram of a method for providing a training-time guardrail for molecular generation, according to at least one embodiment;

FIG. 7 illustrates a flow diagram of a method for providing a guardrail for instruction-tuned molecular generation, according to at least one embodiment;

FIG. 8A illustrates inference and/or training logic, according to at least one embodiment;

FIG. 8B illustrates inference and/or training logic, according to at least one embodiment; and

FIG. 9 illustrates training and deployment of a neural network, according to at least one embodiment.

### DETAILED DESCRIPTION

As discussed herein, the use of generative models in the drug discovery process is increasing, with the potential to accelerate development time and reduce costs over conventional molecular design techniques that have relied on human expertise. However, generative models are also capable of synthesizing molecules that are toxic, harmful, illegal, and/or associated with other undesirable attributes. Further, existing approaches for detecting toxic and/or undesirable molecules are limited in predictive power, the ability to generalize, and/or the ability to scale to large numbers of novel molecules that can be generated using modern deep learning models.

To address the above limitations, the disclosed techniques include various types of guardrails that can be applied independently and/or together to detect and/or prevent the generation of toxic and/or otherwise undesirable molecules using generative models. One type of guardrail uses classifiers to predict probabilities of toxicity, levels of toxicity, binding affinities, docking scores, and/or other affinities/scores/etc. representing measures of undesired attributes for molecules produced by a generative model. These classifiers may be trained using various types of data, including (but not limited to) publicly available binding data, biological assay data, and/or simulation data. The scores outputted by the classifiers may be compared to adjustable thresholds. For example, a low threshold for a score associated with an undesired attribute may be used with malicious use cases to prevent adversarial attacks associated with the output of the generative model. In another example, a user-specified threshold for docking score and/or binding affinity may be used in a non-malicious setting to detect undesirable binding of a given molecule to an off-target. Malicious and/or non-malicious use cases associated with the generative model may be determined using the classifiers based on data such as (but not limited to) the input into the generative model, the output of the generative model, and/or the environment and/or setting used to execute and/or access the generative model. When a given score meets or exceeds a corresponding threshold, the molecule associated with the score may be filtered, flagged, and/or otherwise identified as potentially toxic or undesirable.

A second type of guardrail involves inference-time conditional generation, in which classifiers trained on experimental, simulation, and/or other types of data are integrated into the generative process associated with a given generative model. For example, a classifier may be trained to predict toxicity and/or other undesired attributes in molecules associated with noise samples produced by a diffusion model at various steps in the diffusion and/or reverse diffusion process. During the generation of a new molecule by the diffusion model, the output of the classifier may be used to adjust the distribution of latent variables from which noise is sampled, steer the denoising process that converts the sampled noise into generative output away from potentially toxic and/or undesirable molecules, and/or otherwise guide the process of generating the new molecule in a way that avoids undesirable attributes and/or increases the likelihood of desired attributes.

A third type of guardrail incorporates classifiers trained on experimental, simulation, and/or other types of data into the training of the generative model. For example, a multi-target optimization technique may be used to train the generative model based on multiple objectives, where one or more of the objectives includes non-binding to a dataset of off-targets, lack of toxicity, lack of similarity to an illicit substance, and/or lack of similarity to a protected substance. Losses associated with the objectives may be computed using predictions of toxicity and/or other undesirable attributes outputted by the classifiers from training generative output produced by the generative model. These losses may then be used to train the generative model so that subsequent molecules outputted by the generative model are less likely to bind to any of the off-targets.

A fourth type of guardrail involves processing and/or filtering prompts to a multimodal generative model that is capable of generating molecules and/or synthesis routes for molecules. These prompts may include instructions that describe the types of molecules to be generated, characteristics of the molecules to be generated, synthesis routes for the molecules, and/or other attributes related to the generation of molecules by the generative model. These prompts may also, or instead, include chemical formulas, amino acid sequences, chemical line notations (e.g., simplified molecular-input line-entry system (SMILES) strings, etc.), and/or other representations (e.g., multi-dimensional images, graphs, etc.) of the molecules and/or components of the molecules to be generated. Classifiers that include (but are not limited to) machine learning models, named entity recognition (NER) techniques, parsers, taxonomies, and/or other sources of biomedical information may be used to determine whether or not a given prompt includes instructions, attributes, and/or molecule representations that are associated with toxic, illegal, and/or otherwise undesirable molecules or components of molecules. When a given prompt is determined to include undesirable instructions, attributes, and/or molecule representations, the prompt and/or a user providing the prompt may be blocked, flagged, and/or otherwise prevented from using the generative model to synthesize undesirable molecules.

One technical advantage of the disclosed techniques relative to prior approaches is the ability to detect toxicity and/or other undesirable properties in molecules that are generated using generative models. Accordingly, the disclosed techniques can be used to reduce risk and resource expenditure associated with chemical synthesis, experimental assays, and/or clinical trials involving toxic and/or otherwise undesirable molecules. Another technical advantage of the disclosed techniques is the ability to detect and/or prevent the intentional generation or sharing/presentation of harmful compounds such as those used in biological weapons and/or narcotics. Consequently, the disclosed techniques can be used to safeguard against malicious use of deep learning models that are capable of generating molecules and/or other biological moieties. An additional technical advantage of the disclosed techniques is the ability to identify undesired attributes associated with text-based prompts to generate molecules and/or text-based representations of molecules provided as input into generative models. A further technical advantage of the disclosed techniques is the ability to combine and/or customize different types of guardrails to accommodate use cases, environments, types of users, types of risk, and/or levels of risk associated with molecular generation using generative models.

The above examples are not in any way intended to be limiting. As persons skilled in the art will appreciate, as a general matter, the techniques for automatically generating dialogue flows from unlabeled conversation data can be implemented in any suitable application.

The systems and methods described herein may be used for a variety of purposes, by way of example and without limitation, for use in systems associated with machine control, machine locomotion, machine driving, synthetic data generation, model training, perception, augmented reality, virtual reality, mixed reality, robotics, security and surveillance, simulation and digital twinning, autonomous or semi-autonomous machine applications, deep learning, environment simulation, data center processing, conversational AI, generative AI, light transport simulation (e.g., ray-tracing, path tracing, etc.), collaborative content creation for 3D assets, cloud computing and/or any other suitable applications.

Disclosed embodiments may be comprised in a variety of different systems such as automotive systems (e.g., an infotainment or plug-in gaming/streaming system of an autonomous or semi-autonomous machine), systems implemented using a robot, aerial systems, medial systems, boating systems, smart area monitoring systems, systems for performing deep learning operations, systems for performing simulation operations, systems for performing digital twin operations, systems implemented using an edge device, systems incorporating one or more virtual machines (VMs), systems for performing synthetic data generation operations, systems implemented at least partially in a data center, systems for performing conversational AI operations, systems implementing one or more language models - such as large language models (LLMs), vision language models (VLMs), and/or multi-modal language models that may process text, audio, and/or image data, systems for performing light transport simulation, systems for performing collaborative content creation for 3D assets (e.g., systems or platforms that use universal scene descriptor (USD) data, such as OpenUSD), systems implemented at least partially using cloud computing resources, systems for performing generative AI operations, and/or other types of systems.

### System Overview

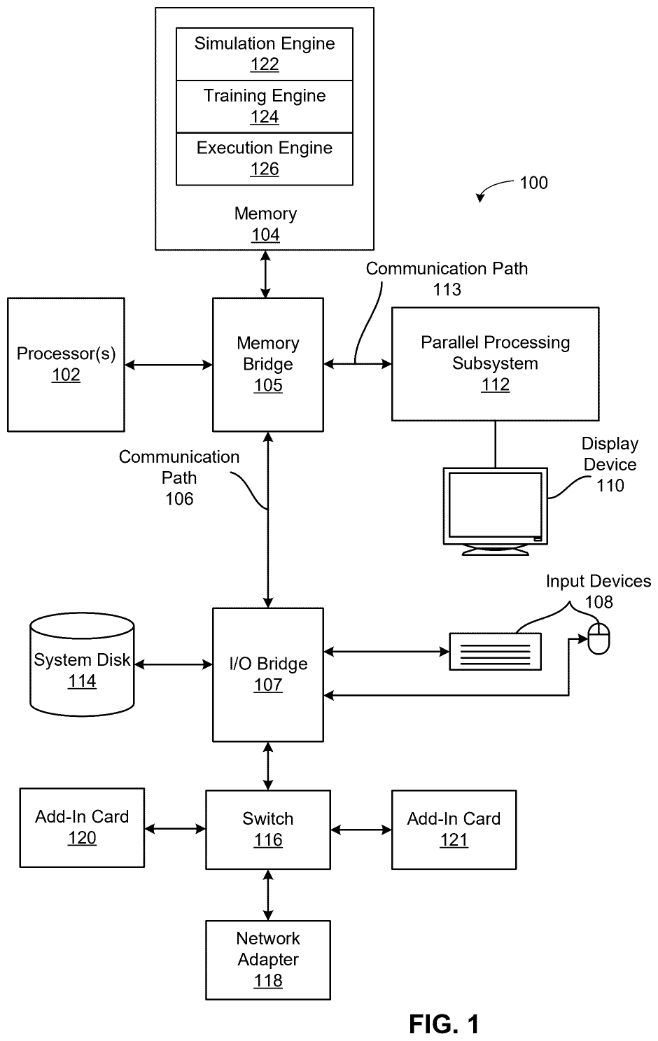

FIG. 1 is a block diagram illustrating a computing system 100 configured to implement one or more aspects of at least one embodiment. In at least one embodiment, computing system 100 may include any type of computing device, including, without limitation, a server machine, a server platform, a desktop machine, a laptop machine, a hand-held/mobile device, a digital kiosk, an in-vehicle infotainment system, a smart speaker or display, a television, and/or a wearable device. In at least one embodiment, computing system 100 is a server machine operating in a data center or a cloud computing environment that provides scalable computing resources as a service over a network.

In various embodiments, computing system 100 includes, without limitation, one or more processors 102 and one or more memories 104 coupled to a parallel processing subsystem 112 via a memory bridge 105 and a communication path 113. Memory bridge 105 is further coupled to an I/O (input/output) bridge 107 via a communication path 106, and I/O bridge 107 is, in turn, coupled to a switch 116.

In one embodiment, I/O bridge 107 is configured to receive user input information from optional input devices 108, such as (but not limited to) a keyboard, mouse, touch screen, sensor data analysis (e.g., evaluating gestures, speech, or other information about one or more uses in a field of view or sensory field of one or more sensors), a VR/MR/AR headset, a gesture recognition system, a steering wheel, mechanical, digital, or touch sensitive buttons or input components, and/or a microphone, and forward the input information to processor(s) 102 for processing. In at least one embodiment, computing system 100 may be a server machine in a cloud computing environment. In such embodiments, computing system 100 may omit input devices 108 and receive equivalent input information as commands (e.g., responsive to one or more inputs from a remote computing device) and/or messages transmitted over a network and received via the network adapter 118. In at least one embodiment, switch 116 is configured to provide connections between I/O bridge 107 and other components of computing system 100, such as a network adapter 118 and various add-in cards 120 and 121.

In at least one embodiment, I/O bridge 107 is coupled to a system disk 114 that may be configured to store content and applications and data for use by processor(s) 102 and parallel processing subsystem 112. In one embodiment, system disk 114 provides non-volatile storage for applications and data and may include fixed or removable hard disk drives, flash memory devices, and CD-ROM (compact disc read-only-memory), DVD-ROM (digital versatile disc-ROM), Blu-ray, HD-DVD (high-definition DVD), or other magnetic, optical, or solid state storage devices. In various embodiments, other components, such as universal serial bus or other port connections, compact disc drives, digital versatile disc drives, film recording devices, and the like, may be connected to I/O bridge 107 as well.

In various embodiments, memory bridge 105 may be a Northbridge chip, and I/O bridge 107 may be a Southbridge chip. In addition, communication paths 106 and 113, as well as other communication paths within computing system 100, may be implemented using any technically suitable protocols, including, without limitation, AGP (Accelerated Graphics Port), HyperTransport, or any other bus or point-to-point communication protocol known in the art.

In at least one embodiment, parallel processing subsystem 112 includes a graphics subsystem that delivers pixels to an optional display device 110 that may be any conventional cathode ray tube, liquid crystal display, light-emitting diode display, and/or the like. In such embodiments, parallel processing subsystem 112 may incorporate circuitry optimized for graphics and video processing, including, for example, video output circuitry. Such circuitry may be incorporated across one or more parallel processing units (PPUs), also referred to herein as parallel processors, included within the parallel processing subsystem 112.

In at least one embodiment, parallel processing subsystem 112 incorporates circuitry optimized (e.g., that undergoes optimization) for general purpose and/or compute processing.

Again, such circuitry may be incorporated across one or more PPUs included within parallel processing subsystem 112 that are configured to perform such general purpose and/or compute operations. In yet other embodiments, the one or more PPUs included within parallel processing subsystem 112 may be configured to perform graphics processing, general purpose processing, and/or compute processing operations. Memor(ies) 104 include at least one device driver configured to manage the processing operations of the one or more PPUs within parallel processing subsystem 112. In addition, memor(ies) 104 include instructions implementing a simulation engine 122, a training engine 124, and an execution engine 126, which can be executed by processor(s) and/or parallel processing subsystem 112.

In various embodiments, parallel processing subsystem 112 may be integrated with one or more of the other elements of FIG. 1 to form a single system. For example, parallel processing subsystem 112 may be integrated with processor(s) 102 and other connection circuitry on a single chip to form a system on a chip (SoC).

Processor(s) 102 may include any suitable processor implemented as a central processing unit (CPU), a graphics processing unit (GPU), an application-specific integrated circuit (ASIC), a field programmable gate array (FPGA), an artificial intelligence (AI) accelerator, a deep learning accelerator (DLA), a parallel processing unit (PPU), a data processing unit (DPU), a vector or vision processing unit (VPU), a programmable vision accelerator (PVA) (which may include one or more VPUs, pixel processing engines (PPEs), and/or direct memory access (DMA) systems), any other type of processing unit, or a combination of different processing units, such as a CPU(s) configured to operate in conjunction with a GPU(s). In general, processor(s) 102 may include any technically feasible hardware unit capable of processing data and/or executing software applications. Further, in the context of this disclosure, the computing elements shown in computing system 100 may correspond to a physical computing system (e.g., a system in a data center or a machine) and/or may correspond to a virtual computing instance executing within a computing cloud.

In at least one embodiment, processor(s) 102 issue commands that control the operation of PPUs. In at least one embodiment, communication path 113 is a Peripheral Component Interconnect Express (PCIe) link, in which dedicated lanes are allocated to each PPU. Other communication paths may also be used. The PPU advantageously implements a highly parallel processing architecture, and the PPU may be provided with any amount of local parallel processing memory (PP memory).

It will be appreciated that the system shown herein is illustrative and that variations and modifications are possible. The connection topology, including the number and arrangement of bridges, the number of processors 102, and the number of parallel processing subsystems 112, may be modified as desired. For example, in at least one embodiment, memor(ies) 104 may be connected to processor(s) 102 directly rather than through memory bridge 105, and other devices may communicate with memor(ies) 104 via memory bridge 105 and processors 102. In other embodiments, parallel processing subsystem 112 may be connected to I/O bridge 107 or directly to processor(s) 102, rather than to memory bridge 105. In still other embodiments, I/O bridge 107 and memory bridge 105 may be integrated into a single chip instead of existing as one or more discrete devices. In certain embodiments, one or more components shown in FIG. 1 may not be present. For example, switch 116 may be eliminated, and network adapter 118 and add-in cards 120, 121 would connect directly to I/O bridge 107. Further, in certain embodiments, one or more components shown in FIG. 1 may be implemented as virtualized resources in a virtual computing environment, such as a cloud computing environment. In particular, the parallel processing subsystem 112 may be implemented as a virtualized parallel processing subsystem in at least one embodiment. For example, the parallel processing subsystem 112 may be implemented as a virtual graphics processing unit(s) (vGPU(s)) that renders graphics on a virtual machine(s) (VM(s)) executing on a server machine(s) whose GPU(s) and other physical resources are shared across one or more VMs.

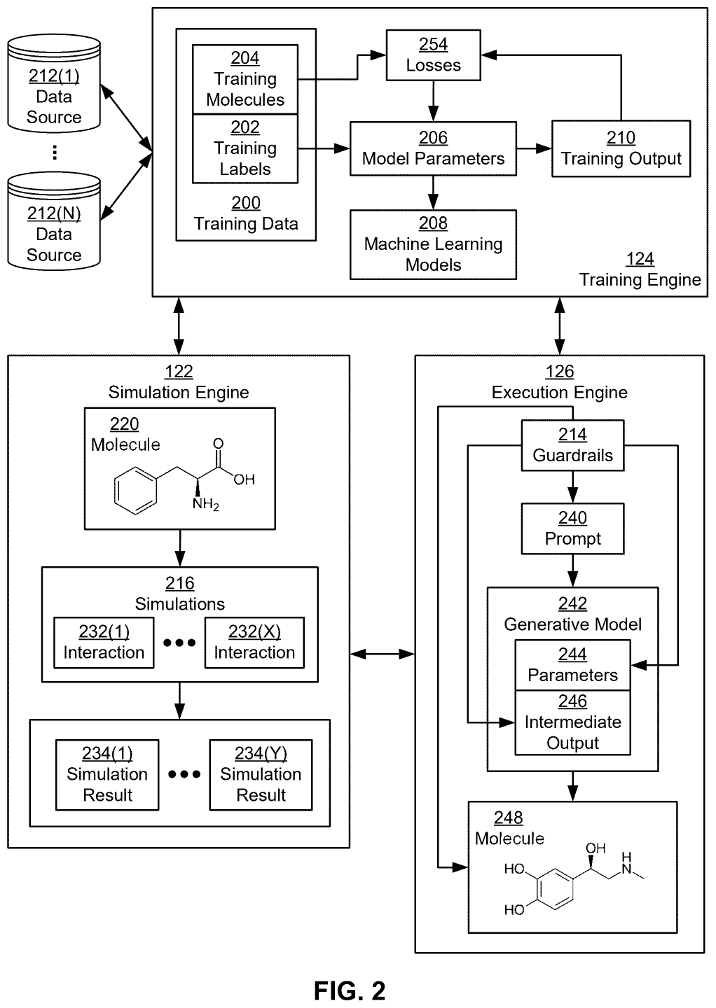

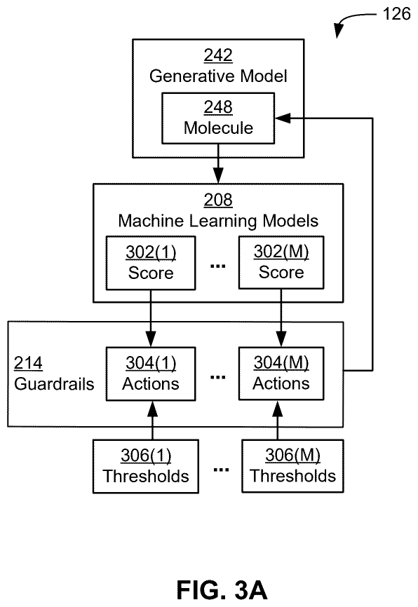

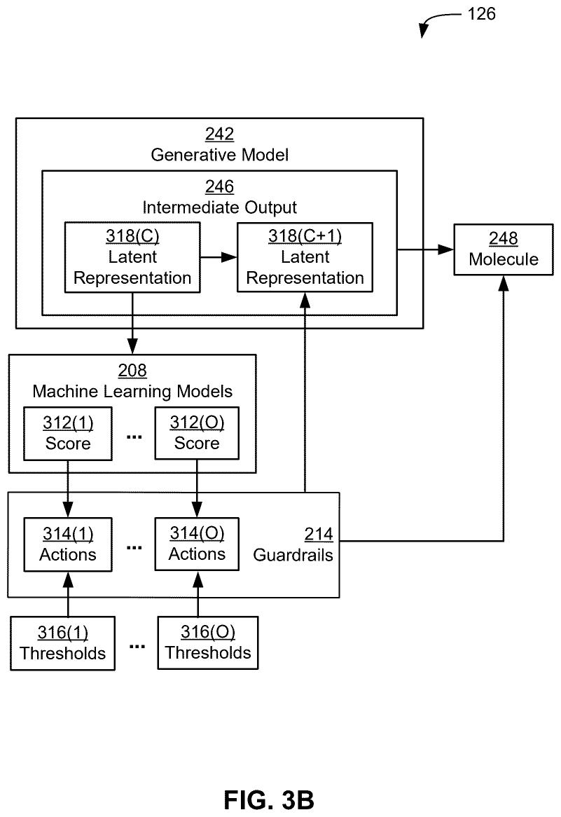

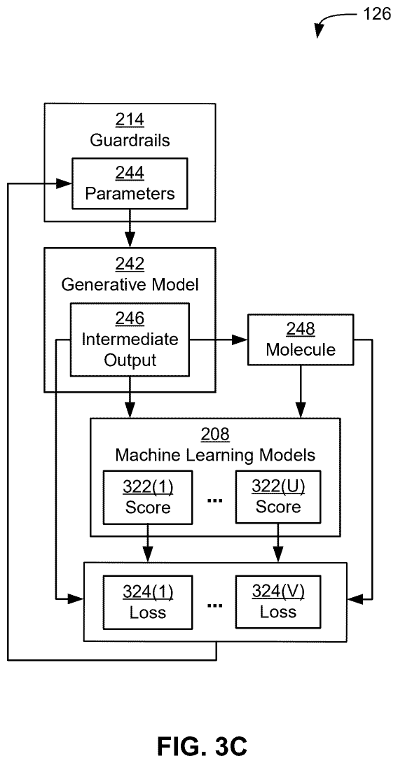

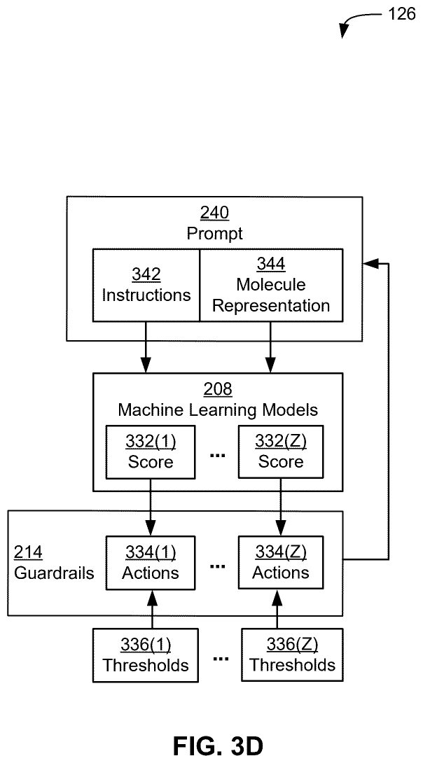

In some embodiments, simulation engine 122, training engine 124, and execution engine 126 include functionality to implement various types of guardrails that can be applied independently and/or together to detect and/or prevent the generation of toxic and/or otherwise undesirable molecules using generative models. These guardrails may involve the use of classifiers that are trained using various types of data, including (but not limited to) publicly available binding data, biological assay data, and/or simulation data generated by simulation engine 122. The trained classifiers may be used to predict probabilities of toxicity, levels of toxicity, binding affinities, docking scores, and/or other scores representing measures of undesired attributes associated with molecules produced by a generative model, intermediate output that is used by the generative model to generate the molecules, and/or input into the generative model. These scores may be used to filter the generated molecules and/or input, guide the generation of subsequent intermediate output by the generative model, train the generative model, and/or otherwise reduce risk and/or resource expenditure associated with intentional and/or unintentional generation of molecules with undesired attributes by the generative model. Simulation engine 122, training engine 124, and execution engine 126 are described in further detail below with respect to FIGS. 2 and 3A-3D.

### Guardrails for Molecular Generation

FIG. 2 is a more detailed illustration of simulation engine 122, training engine 124, and execution engine 126 of FIG. 1, according to at least one embodiment. As discussed herein, simulation engine 122, training engine 124, and execution engine 126 are configured to incorporate one or more machine learning models 208 into various guardrails 214 associated with the generation of a molecule(s) 248 by a generative model 242. Each of these components is described in further detail below.

Simulation engine 122 performs one or more simulations 216 of interactions 232(1)-232(X) (each of which is referred to individually herein as interaction 232) associated with a molecule 220. For example, simulation engine 122 may use molecular dynamics techniques, free energy perturbation (FEP) calculations, molecular mechanics/generalized Born surface area (MM/GBSA) techniques, machine learning techniques, genetic techniques, torsional searches, and/or other techniques to conduct simulations 216 that analyze forces, motions, and/or other attributes of interactions 232 among particles within molecule 220, between the particles of molecule 220 and/or particles of other molecules, and/or between molecule 220 and the other molecules.

After a given simulation is complete, simulation engine 122 generates one or more simulation results 234(1)-234(Y) (each of which is referred to individually herein as simulation result 234) based on interactions 232 modeled in the simulation. For example, simulation engine 122 may include, in a set of simulation results 234 for a given simulation involving molecule 220 and a receptor (e.g., a protein and/or biopolymer), a docking, binding affinity, ligand flexibility, receptor flexibility, free energy, binding mode, pose, docking accuracy, and/or other attributes associated with interactions between molecule 220 and the receptor.

In some embodiments, simulation engine 122 uses a hierarchy of simulations 216 associated with a given molecule 220 to generate corresponding simulation results 234. Within this hierarchy, an initial round of less accurate and low resource overhead simulations 216 may be performed to provide a preliminary understanding of the behavior of a given molecule 220.

This initial round of simulations 216 may include polynomial form, neural network, and/or other approximations of complex equations describing interactions 232. If simulation results 234 associated with this initial round of simulations 216 are inconclusive, one or more additional rounds of simulations 216 with higher accuracy and/or resource overhead may be performed.

These additional round(s) of simulations 216 may include more complete representations of interactions 232 and/or equations describing interactions 232. Thus, simulations 216 may selectively be performed based on the complexity or uncertainty of the behavior of molecule 220.

Simulation engine 122 may additionally perform simulations 216 and generate corresponding simulation results 234 for various types of molecules. For example, simulation engine 122 may perform simulations 216 of interactions 232 associated with one or more classes of molecules (e.g., small molecules, biologics, etc.), molecules with a certain structure, molecules with a certain composition, molecules from a database, a curated set of molecules, and/or other types of molecules.

Training engine 124 trains one or more machine learning models 208 using training data 200 derived from simulation results 234 of simulations 216 by simulation engine 122 and/or one or more additional data sources 212(1)-212(N) (each of which is referred to individually herein as data source 212). Data sources 212 include (but are not limited to) databases of binding data, clinical trial results, in vitro and/or in vivo studies, and/or other types of experimental data.

In some embodiments, data from data sources 212 includes results of biological assays associated with a set of molecules. These results may characterize the biological activity, efficacy, toxicity, pharmacokinetics, pharmacodynamics, therapeutic windows, and/or other attributes of the molecules. The biological assays may include in vitro assays such as cell viability tests, enzyme inhibition assays, receptor binding assays, and gene expression profiling. The biological assays may also, or instead, include in vivo assays such as animal studies that are used to assess the therapeutic and toxic effects of molecules in a living organism. Outputs from multiple assays may also be combined.

As shown in FIG. 2, training data 200 includes a set of training molecules 204 and a set of training labels 202. Training molecules 204 include representations of molecules associated with simulations 216 and/or biological assays. For example, training molecules 204 may include chemical formulas, skeletal structures, graphs, strings, embeddings, latent representations, and/or other representations of small organic compounds, peptides, nucleic acids, carbohydrates, complex natural products, drugs, experimental compounds, molecules with well-characterized pharmacological profiles, and/or other types of molecules of interest. Training molecules 204 may be selected based on relevance to specific therapeutic areas, structural diversity, representation of a particular chemical space of interest, and/or other factors.

Training labels 202 may represent properties or behaviors of training molecules 204, as determined using simulations 216, biological assays, and/or data from data sources 212. For example, training labels 202 may include binary classifications of toxicity or non-toxicity, binding or non-binding to a target and/or off-target, and/or other attributes associated with training molecules 204. Training labels 202 may also, or instead, include quantitative measures of toxicity levels, binding affinities, and/or other types of biological activity. Training labels 202 may also, or instead, include multi-class labels that categorize training molecules 204 into different classes based on pharmacological effects, mechanisms of action, safety profiles, and/or other attributes.

Machine learning models 208 are configured to perform classification and/or other types of predictive tasks based on training molecules 204. For example, machine learning models 208 may include tree-based models such as decision trees, random forests, and gradient-boosted trees; feedforward neural networks, convolutional neural networks (CNNs), recurrent neural networks (RNNs), residual neural networks, long short-term memory networks (LSTMs), graph neural networks, transformer neural networks, and/or other types of neural networks; and/or support vector machines (SVMs), logistic regression models, hierarchical models, ensemble models, Bayesian networks, naïve Bayes classifiers, and/or other types of model architectures. Machine learning models 208 may also, or instead, include rules, filters, heuristics, logic programming, semantic nets, search techniques, named entity recognition techniques, and/or other symbolic models of molecules, structures within molecules, and/or attributes of molecules.

During training of machine learning models 208, training engine 122 inputs training molecules 204 into machine learning models 208. Training engine 122 uses model parameters 206 (e.g., neural network weights) of machine learning models 208 to process the inputted training molecules 204 and obtains training output 210 that includes predictions of training labels for the inputted training molecules 204 from one or more layers, blocks, or components of machine learning models 208. Training engine 124 computes one or more losses 254 (e.g., cross entropy loss, triplet loss, contrastive loss, etc.) between training output 210 and the corresponding training labels 202. Training engine 124 then uses a training technique (e.g., gradient descent and backpropagation) to iteratively update model parameters 206 of machine learning models 208 in a way that reduces losses 254. After training of a given machine learning model is complete, that machine learning model is capable of predicting one or more training labels 202 for one or more training molecules 204, given input that includes representations of those training molecules 204.

In one or more embodiments, individual machine learning models 208 are targeted toward specific attributes of interest, types of molecules, and/or other criteria. For example, machine learning models 208 may include multiple classifiers that predict toxicity, levels of toxicity, and/or other attributes related to toxicity for a specific toxicity profile. In another example, machine learning models 208 may include multiple classifiers that predict various attributes for a given target and/or off-target. In a third example, machine learning models 208 may include a different classifier and/or ensemble of classifiers for a given attribute of interest to be predicted, type of molecule (e.g., small molecules, biologics, peptides, nucleic acids, etc.), biological target or pathway, therapeutic area (e.g., oncology, neurology, cardiology, etc.), and/or another factor associated with the generation and/or use of molecules. In a fourth example, machine learning models 208 may be used to perform hierarchical classification, in which one or more classifiers initially categorize molecules into broad categories and/or buckets of attributes and additional levels of classifiers generate more detailed predictions within each category and/or bucket.

Execution engine 126 uses the trained machine learning models 208 to implement various types of guardrails 214 associated with the generation of a given molecule 248 by a generative model 242. More specifically, execution engine 126 may use a diffusion model, reinforcement learning model, recurrent neural network (RNN), transformer neural network, variational autoencoder (VAE), large language model (LLM), generative adversarial network (GAN), normalizing flow model, diffusion normalizing flow model, multimodal model, and/or another type of generative model 242 to generate molecule 248 and/or a synthesis route for molecule 248 over one or more iterations of a molecular design process. During a given iteration, execution engine 126 may input molecular structures, desired targets and/or physicochemical properties, constraints, and/or other data related to a given molecule 248 to be generated into generative model 242. Execution engine 126 may execute generative model 242 based on this inputted data to produce one or more molecules (e.g., molecule 248). Execution engine 126 may also, or instead, modify and/or supplement the generated molecule(s) using databases of known molecules, user input, and/or manipulations of atoms, bonds, functional groups, stereochemistry, and/or other components of the generated molecules. Execution engine 126 may then use the generated, supplemented, and/or modified molecules as a “seed” for the next iteration of the molecular design process and/or additional data that is inputted into generative model 242 during the next iteration. Execution engine 126 may also, or instead, include one or more molecules 248 generated by generative model 242 in a set of drug candidates.

During each iteration and/or certain iterations of the molecular design process, execution engine 126 uses machine learning models 208 to detect toxicity and/or other undesired attributes in representations of a given molecule 248 (or a portion of a given molecule 248) outputted by generative model 242. For example, execution engine 126 may use the output of machine learning models 208 to flag and/or filter molecule 248, exclude molecule 248 from subsequent iterations of the molecular design process, adjust the process by which generative model 242 produces molecules, and/or implement other guardrails 214 to reduce risk and resource expenditure associated with chemical synthesis, experimental assays, and/or clinical trials involving toxic and/or otherwise undesirable molecules.

In some embodiments, guardrails 214 include the use of machine learning models 208 to perform inference-time detection of toxicity, illegality, binding to an off-target, and/or other undesired attributes in a given molecule 248 (or portion of that molecule 248) generated by generative model 242. As described in further detail below with respect to FIG. 3A, this inference-time detection may be customized for specific types of molecules, molecular generation use cases, standards of care, and/or other types of scenarios associated with the generation and/or use of biological moieties. When an undesired attribute is detected in one or more portions of molecule 248, the portion(s) may be filtered from subsequent iterations of the molecular design process, blocked from being outputted to one or more users, flagged for review, and/or otherwise processed to mitigate risks associated with the undesired attribute.

Guardrails 214 may also, or instead, include the use of machine learning models 208 to perform inference-time conditional generation by generative model 242. As described in further detail below with respect to FIG. 3B, this inference-time conditional generation may involve the use of one or more machine learning models 208 to predict toxicity and/or other undesired attributes in intermediate output 246 of generative model 242, such as latent representations of a given molecule 248 produced by a diffusion model at various steps in the diffusion and/or reverse diffusion process and/or by a VAE, GAN, and/or another type of latent variable model. The output of machine learning models 208 may be used to adjust the distribution of latent variables from which noise is sampled, steer the denoising process that converts the sampled noise into generative output away from potentially toxic and/or undesirable molecules, and/or otherwise guide the process of generating a given molecule 248 in a way that avoids undesirable attributes and/or increases the likelihood of desired attributes.

Guardrails 214 may also, or instead, incorporate machine learning models 208 into the training process for generative model 242. As described in further detail below with respect to FIG. 3C, one or more losses may be computed using predictions of toxicity and/or other undesirable attributes generated by machine learning models 208 from training generative output produced by generative model 242. These losses may then be used to update parameters 244 of generative model 242 so that subsequent molecules outputted by generative model 242 are less likely to include these undesirable attributes.

Guardrails 214 may also, or instead, use machine learning models 208 to process and/or filter a prompt 240 to a multimodal generative model 242 that is capable of generating molecules and/or instructions for synthesizing molecules. This prompt 240 may include descriptions of the types of molecules to be generated, characteristics of the molecules to be generated, synthesis routes for the molecules, and/or other instructions related to the generation of molecules by the generative model. Prompt 240 may also, or instead, include chemical formulas, amino acid sequences, chemical line notations (e.g., simplified molecular-input line-entry system (SMILES) strings, etc.), and/or other representations (e.g., multi-dimensional images, graphs, etc.) of the molecules and/or components of the molecules to be generated. As described in further detail below with respect to FIG. 3D, when a given prompt 240 is determined to include instructions and/or molecule representations that are associated with toxic molecules, illicit substances, and/or otherwise undesirable attributes, that prompt 240 and/or a user providing that prompt 240 may be blocked, flagged for review, and/or otherwise prevented from using prompt 240 to generate undesirable molecules.

FIG. 3A illustrates how machine learning models 208 of FIG. 1 are used to provide inference-time guardrails 214 for molecular generation, according to at least one embodiment. As shown in FIG. 3A, machine learning models 208 are used to generate a set of scores 302(1)-302(M) (each of which is referred to individually herein as score 302) from input that includes a given molecule 248 outputted by generative model 242. Each score 302 represents a predicted measure of a different undesired attribute associated with molecule 248. For example, scores 302 may represent predictions of toxicity, toxicity levels, binding to an off-target, binding affinity to an off-target, bioavailability, metabolic stability, potential for drug-drug interactions, illegality, regulatory restrictions, intellectual property restrictions, and/or other attributes of interest associated with a certain type of molecule 248 and/or an application of molecule 248.

Execution engine 126 compares each score 302 with a set of one or more corresponding thresholds 306(1)-306(M) (each of which is referred to individually herein as thresholds 306).

Each set of thresholds 306 for a given score 302 may specify a minimum and/or maximum acceptable value, a range of acceptable values, a set of “acceptable” values, a set of “unacceptable” values, and/or other representations of values for one or more attributes represented by that score 302. For example, thresholds 306 may include a minimum and/or maximum value for a likelihood of binding, binding affinity, binding selectivity, enzyme inhibition, enzyme activation, level of toxicity, bioavailability, metabolic stability, likelihood of a drug-drug interaction, efficacy, potency, and/or another attribute associated with a given score 302 outputted by machine learning models 208. In another example, thresholds 306 may include acceptable and/or unacceptable values for scores 302 representing categorical attributes such as (but not limited to) binding to a target or off-target, side effects, drug-drug interactions, toxicity or non-toxicity, restricted (e.g., patented, illegal, non-compliant with regulations, etc.) molecules and/or substances, market status (e.g., investigational, approved, withdrawn, etc.), pharmacokinetic properties, pharmacodynamic properties, and/or biological activity profiles. In a third example, thresholds 306 may include minimum values, maximum values, ranges and/or sets of acceptable values, and/or ranges and/or sets of unacceptable values for sums, averages, counts, and/or other aggregations and/or combinations of scores 302.

In some embodiments, thresholds 306 are set and/or adjusted based on requirements, constraints, and/or other factors associated with the generation and/or use of molecule 248. For example, a given score 302 for toxicity may be associated with multiple thresholds 306 representing levels of risk associated with different use cases. A first threshold may be set for malicious use cases, such as the use of generative model 242 to design biochemical weapons, toxic and/or poisonous substances, and/or illicit substances. A second threshold may be set for non-malicious use cases, such as the use of generative model 242 to perform drug discovery. The first threshold may represent a lower tolerance level for toxicity and/or likelihood of toxicity than the second threshold to prevent adversarial attacks and/or other malicious use of molecule 248 and/or generative model 242. Different thresholds 306 for a given score 302 and/or set of scores may also be used to reflect different sets of requirements, regulatory guidelines, standards of care, stages of molecular generation (e.g., the number of iterations performed during a molecular generation process, etc.), levels of risk (e.g., mild toxicity, severe toxicity, lethality, etc.), types of attributes (e.g., specific targets, off-targets, pathways, treatments, etc.), user preferences, industry benchmarks, tradeoffs between desired and/or undesired attributes, available therapeutics, patient lifespans, and/or other criteria or considerations associated with generation and/or use of molecule 248. These criteria and/or considerations may be predefined for a given instance of generative model 242, molecular design process involving generative model 242, user, and/or another environment or setting in which generative model 242 may be accessed and/or used. These criteria and/or settings may also, or instead, be predicted using one or more machine learning models 208, as described in further detail below.

If a given score 302 and/or set of scores 302 falls outside a range and/or set of acceptable values denoted by a corresponding set of thresholds 306, execution engine 126 performs one or more actions 304(1)-304(M) (each of which is referred to individually herein as actions 304) to implement guardrails 214 that mitigate the risk associated with the corresponding attribute. Actions 304 may include (but are not limited to) flagging molecule 248 for further review, excluding molecule 248 from subsequent iterations of the molecular design process, omitting molecule 248 from a user interface, and/or modifying the structure of molecule 248 to reduce the probability or impact of the undesired attribute.

Actions 304 may also be determined and/or performed in a way that reflects the level of risk, importance, and/or other considerations associated with the corresponding thresholds 306 and/or use cases. For example, a certain score 302 that exceeds a threshold representing a high level of toxicity and/or risk may result in actions 304 that include (but are not limited to) filtering molecule 248 from subsequent iterations of a molecular design and/or drug discovery process, while a certain score 302 that exceeds a threshold representing a lower level of toxicity and/or risk may result in actions 304 that include (but are not limited to) flagging molecule 248 for user review and/or reducing the priority of molecule 248 in a drug discovery process.

In another example, a certain score 302 that exceeds a threshold for toxicity and/or risk for a malicious use case may result in actions 304 that include (but are not limited to) blocking the output of molecule 248 to a user, blocking of the user from subsequent use of generative model 242, and/or generation of a security alert related to a potential adversarial attack. Conversely, a certain score 302 that exceeds a threshold for toxicity and/or risk for a non-malicious use case may cause a notification of the exceeded threshold for the corresponding molecule 248 to be outputted to a user (e.g., in a user interface such as a graphical user interface) involved in the molecular design process and/or deprioritization of molecule 248 in the molecular design process.

In a third example, a certain score 302 that indicates that a corresponding molecule 248 falls within a restricted category (e.g., a controlled substance, a patented molecule, a molecule that does not comply with environmental and/or safety regulations, etc.) may result in actions 304 such as (but not limited to) omitting that molecule 248 from subsequent molecular design and/or drug discovery processes and/or flagging molecule 248 and/or a user involved in generation of molecule 248 for review.

In a fourth example, actions 304 may include generating a report and/or profile of a given molecule 248 based on the results of comparisons between scores 302 and the corresponding thresholds 306. The report and/or profile may identify scores 302 that fall within ranges and/or sets of acceptable values, scores 302 that do not fall within ranges and/or sets of acceptable values, the deviation of a given score 302 from a corresponding threshold, attributes represented by thresholds 306 that are not met by the corresponding scores 302, attributes represented by thresholds 306 that are met by the corresponding scores, and/or other information that can be used to characterize the predicted behavior and/or properties of molecule 248. This report and/or profile may be outputted to one or more users involved in the molecular design process to assist the users in selecting (or excluding from selection) molecules as seeds for subsequent iterations of the molecular design process, drug candidates, and/or other uses.

FIG. 3B illustrates how machine learning models 208 of FIG. 1 are used to provide guardrails 214 for conditional molecular generation, according to at least one embodiment. As shown in FIG. 3B, input into machine learning models 208 includes a given latent representation 318(C) that is generated as intermediate output 246 of generative model 242. For example, latent representation 318(C) may correspond to a partially denoised representation of a given molecule 248 that is produced by a diffusion-based generative model 242 during the C^{th} iteration of a denoising process that converts a noise sample into molecule 248. In another example, latent representation 318(C) may be outputted by one or more layers of an encoder and/or decoder in a VAE, GAN, multimodal generative model 242, and/or another type of generative model 242 that includes a latent space and functionality to generate molecules.

Machine learning models 208 convert the inputted latent representation 318(C) into a set of scores 312(1)-312(O) (each of which is referred to individually herein as score 312). Like scores 302 of FIG. 3A, scores 312 represent predicted measures of different undesired attributes associated with molecule 248. For example, scores 312 may represent predictions of toxicity, toxicity levels, binding to an off-target, binding affinity to an off-target, bioavailability, metabolic stability, potential for drug-drug interactions, and/or other attributes of interest associated with a certain type of molecule 248 and/or an application of molecule 248.

Unlike machine learning models 208 of FIG. 3A that generate predictions related to undesired attributes based on molecules and/or portions of molecules, machine learning models 208 of FIG. 3B are used to generate predictions related to undesired attributes based on latent and/or noisy representations of molecules. For example, machine learning models 208 of FIG. 3B may include equivariant graph neural networks, transformer neural networks, and/or other types of neural networks and/or machine learning models. These neural networks may include layers, blocks, and/or other elements that are shared with generative model 242. These neural networks may be trained to predict attributes of interest in a given molecule 248, given input that includes one or more latent representations (e.g., latent representation 318(C)) of that molecule 248.

Execution engine 126 compares each score 312 with a set of one or more corresponding thresholds 316(1)-316(O) (each of which is referred to individually herein as thresholds 316).

Each set of thresholds 316 represents a minimum and/or maximum acceptable value, a range of acceptable values, one or more “acceptable” values, one or more “unacceptable” values, and/or other representations of values for one or more attributes associated with a corresponding score 312 and/or set of scores 312. For example, thresholds 316 may include a minimum and/or maximum value for a likelihood of binding, binding affinity, binding selectivity, enzyme inhibition, enzyme activation, level of toxicity, bioavailability, metabolic stability, likelihood of a drug-drug interaction, efficacy, potency, and/or another attribute associated with a given score 312 outputted by machine learning models 208. In another example, thresholds 316 may include acceptable and/or unacceptable values for scores 312 representing categorical attributes such as (but not limited to) binding to a target or off-target, side effects, drug-drug interactions, toxicity or non-toxicity, restricted (e.g., patented, illegal, non-compliant with regulations, etc.) molecules and/or substances, market status (e.g., investigational, approved, withdrawn, etc.), pharmacokinetic properties, pharmacodynamic properties, and/or biological activity profiles. In a third example, thresholds 316 may include minimum values, maximum values, ranges and/or sets of acceptable values, and/or ranges and/or sets of unacceptable values for sums, averages, counts, and/or other aggregations and/or combinations of scores 312.

As with thresholds 306 of FIG. 3A, thresholds 316 are set and/or adjusted based on requirements, constraints, and/or other factors associated with the generation and/or use of molecule 248. For example, a given score 312 for toxicity may be associated with multiple thresholds 316 representing levels of risk associated with different use cases. A first threshold may be set for malicious use cases, such as the use of generative model 242 to design biochemical weapons, toxic and/or poisonous substances, and/or illicit substances. A second threshold may be set for non-malicious use cases, such as the use of generative model 242 to perform drug discovery. The first threshold may represent a lower level tolerance for toxicity and/or likelihood of toxicity than the second threshold to prevent adversarial attacks and/or other malicious use of molecule 248 and/or generative model 242. Different thresholds 316 for a given score 312 and/or set of scores 312 may also reflect different sets of requirements, regulatory guidelines, standards of care, stages of molecular generation (e.g., the number of iterations performed during a molecular generation process, etc.), levels of risk (e.g., mild toxicity, severe toxicity, lethality, etc.), types of attributes (e.g., specific targets, off-targets, pathways, treatments, etc.), user preferences, industry benchmarks, tradeoffs between desired and/or undesired attributes, available therapeutics, patient lifespans, and/or other criteria or considerations associated with generation and/or use of molecule 248.

If a given score 312 or set of scores 312 falls outside a range and/or set of acceptable values denoted by a corresponding set of thresholds 316, execution engine 126 performs one or more actions 314(1)-314(O) (each of which is referred to individually herein as actions 314) to implement guardrails 214 that mitigate the risk associated with the corresponding attribute. In some embodiments, actions 314 include modifying the way in which generative model 242 converts latent representation 318(C) into a subsequent latent representation 318(C+1). For example, generative model 242 may include a diffusion model that removes noise from latent representation 318(C) to produce latent representation 318(C+1). When a given score 312 associated with latent representation 318(C) exceeds a corresponding threshold 316 for binding to an off-target, execution engine 126 may condition the reverse stochastic differential equation (SDE) used by the diffusion model to convert latent representation 318(C) into latent representation 318(C+1) on a label and/or another type of input associated with the exceeded threshold 316.

In another example, generative model 242 may include a VAE, GAN, and/or another type of latent variable model. When a given score 312 associated with latent representation 318(C) exceeds a corresponding threshold 316 for toxicity and/or a certain level of toxicity, execution engine 126 may generate latent representation 318(C+1) by combining a class and/or another representation associated with score 312 and/or threshold 316 with latent representation 318(C), projecting latent representation 318(C) onto a subspace that is not associated with an undesired attribute corresponding to score 312 and/or threshold 316, “reweighting” latent representation 318(C) using the representation, performing gradient-based optimization of latent representation 318(C) using the representation, and/or otherwise using the representation to modify latent representation 318(C). By converting latent representation 318(C) into latent representation 318(C+1) based on score 312 and/or threshold 316, execution engine 126 may reduce the probability of latent representation 318(C+1) and/or the resulting molecule 248 falling outside the range and/or set of acceptable values represented by threshold 316.

Continuing with the above example, execution engine 126 may also, or instead, “filter” out latent representation 318(C) and use generative model 242 to resample a new latent representation 318(C+1) from the latent space. Execution engine 126 may repeat this process until a given latent representation results in scores 312 that do not correspond to unacceptable values for the corresponding attributes.

After a new latent representation (e.g., latent representation 318(C+1)) is generated based on scores 312 and/or thresholds 316, execution engine 126 may repeat the process of using machine learning models 208 to compute scores 312 for the new latent representation, comparing scores 312 to the corresponding thresholds 316, and performing one or more actions 314 based on the results of the comparison. Consequently, execution engine 126 may “steer” the generative process associated with generative model 242 away from latent representations and/or other intermediate output 246 that can result in a corresponding molecule 248 with undesired attributes.

Actions 314 may also, or instead, be applied to molecule 248 produced by generative model 242 from intermediate output 246. For example, execution engine 126 may obtain latent representation 318(C) as a partially denoised sample generated by a diffusion-based generative model 242 during a penultimate denoising step (or another denoising step preceding the generation of molecule 248). Execution engine 126 may also use generative model 242 to perform one or more additional denoising steps that convert latent representation 318(C) into molecule 248. Execution engine 126 may additionally use machine learning models 208 to generate scores 312 for latent representation 318(C). If a given score 312 does not fall within a range or set of acceptable values associated with these thresholds 316, execution engine 126 may iteratively optimize molecule 248 based on that score 312 and/or thresholds 316. During this iterative optimization of molecule 248, execution engine 126 may compute one or more losses based on score 312 and/or thresholds 316. Execution engine 126 may also use a coordinate descent technique, gradient descent technique, and/or another type of optimization technique to iteratively update molecule 248 in a way that reduces the computed losses. After a given update to molecule 248 is made, execution engine 126 may compute a new latent representation 318(C+1) of molecule 248 and repeat the process using this new latent representation 318(C+1) until the losses fall below a threshold, score 312 falls within a set or range of acceptable values denoted by thresholds 316, and/or other criteria are met.

In another example, execution engine 126 may use an embedding model, VAE, multimodal model, forward diffusion process, and/or another technique to convert a given molecule 248 outputted by generative model 242 into a corresponding latent representation 318(C). Execution engine 126 may use machine learning models 208 to generate scores 312 for latent representation 318(C). If a given score 312 does not fall within a range or set of acceptable values associated with a corresponding set of thresholds 316, execution engine 126 may use a coordinate descent technique, gradient descent technique, and/or another type of optimization technique to iteratively optimize molecule 248 based on losses associated with score 312 and/or thresholds 316 until the losses fall below a threshold, score 312 falls within a set or range of a set of acceptable values denoted by thresholds 316, and/or other criteria are met.

FIG. 3C illustrates how machine learning models 208 of FIG. 1 are used to provide training-time guardrails 214 for molecular generation, according to at least one embodiment. As mentioned herein, these training-time guardrails 214 may be used to guide the training of generative model 242 in a way that reduces the likelihood of toxicity, binding to off-targets, and/or other undesirable attributes in molecules outputted by generative model 242.

As shown in FIG. 3C, input into machine learning models 208 includes intermediate output 246 produced by generative model 242 during training of generative model 242. This intermediate output 246 may include (but is not limited to) latent representations (e.g., latent representations 318(C)-318(C+1)) and/or other types of representations of a given molecule 248 that can be generated by generative model 242, as described above with respect to FIG. 3B.

Input into machine learning models 208 also, or instead, includes the resulting molecule 248 outputted by generative model 242 from one or more latent representations and/or other intermediate output 246.

As with machine learning models 208 of FIGS. 3A-3B, machine learning models 208 of FIG. 3C are used to generate scores 322(1)-322(U) (each of which is referred to individually herein as score 322) representing predicted measures of different undesired attributes, given the corresponding inputted intermediate output 246 and/or molecule 248. For example, scores 322 may represent predictions of toxicity, toxicity levels, binding to an off-target, binding affinity to an off-target, bioavailability, metabolic stability, potential for drug-drug interactions, and/or other attributes of interest associated with a certain type of molecule 248, an application of molecule 248, and/or intermediate output 246 that is converted into molecule 248 by generative model 242.

Execution engine 126 uses intermediate output 246 and/or molecule 248 to computes a set of losses 324(1)-324(V) (each of which is referred to individually herein as loss 324) that can be used to train and/or modify the operation of generative model 242. For example, execution engine 126 may compute one or more losses 324 as a mean squared error, cross-entropy loss, reconstruction loss, and/or another measure of error between a given molecule 248 outputted by generative model 242 and a corresponding ground-truth molecule. Execution engine 126 may also, or instead, compute one or more losses 324 as a perceptual loss, Kullback-Leibler (KL) divergence, and/or another measure of error or divergence between (i) individual latent representations and/or a distribution of latent representations in intermediate output 246 of generative model 242 and (ii) ground-truth latent representations and/or a “target” distribution of latent representations.

In some embodiments, one or more losses 324 are computed using scores 322 outputted by machine learning models 208 from intermediate output 246 and/or molecule 248. For example, execution engine 126 may condition some or all latent representations and/or distributions of latent representation used to compute losses 324 based on predictions of toxicity, binding to off-targets, binding affinities, probabilities, and/or other values corresponding to some or all scores 322 outputted by machine learning models 208. In another example, execution engine 126 may include representations of one or more scores 322 predicted by machine learning models 208 from intermediate output 246 and/or molecule 248 in one or more losses 324. In a third example, execution engine 126 may use one or more machine learning models 208 as discriminators that identify toxicity, binding to off-targets, and/or other undesired attributes in intermediate output 246 and/or molecule 248. The output of these discriminators may then be incorporated into one or more losses 324.

Execution engine 126 also uses the computed losses 324 to implement guardrails 214 that update parameters 244 of generative model 242 in a way that mitigates risks associated with attributes represented by scores 322. For example, execution engine 126 may use multiple-gradient descent, multi-task learning, and/or multi-objective optimization to iteratively adjust parameters 244 of generative model 242 in a way that reduces multiple losses 324 associated with intermediate output 246, molecule 248, and/or scores 322. During this iterative training of generative model 242, execution engine 126 may combine losses 324 into a single loss function (e.g., using a weighted combination of losses 324), train generative model 242 over multiple stages using different types of losses 324, and/or use other techniques to train generative model 242 using these losses 324.

Execution engine 126 may also, or instead, use one or more losses 324 to optimize the generation of additional intermediate output 246 and/or molecule 248 by generative model 242. For example, execution engine 126 may use losses 324 to iteratively optimize latent representations generated by generative model 242 and/or the resulting molecule 248, as discussed above with respect to FIG. 3C.

FIG. 3D illustrates how machine learning models 208 of FIG. 1 are used to provide guardrails 214 for instruction-tuned molecular generation, according to at least one embodiment. As shown in FIG. 3D, input into machine learning models 208 includes one or more portions of a prompt 240 into an LLM, multimodal model, and/or another type of generative model 242. These portion(s) may include one or more instructions 342 associated with generating a molecule. For example, instructions 342 may include desired attributes of the molecule, synthesis routes of the molecule, and/or other text-based conditions associated with generation of the molecule.

These portion(s) of prompt 240 may also, or instead, include a molecule representation 344 of the molecule. For example, molecule representation 344 may include (but is not limited to) a chemical formula, amino acid sequence, SMILE string, image, graph, and/or another representation of the structure and/or composition of the molecule.

In one or more embodiments, execution engine 126 parses prompt 240 into one or more instructions 342 and/or molecule representation 344. For example, execution engine 126 may use natural language processing (NLP) techniques to extract an instruction of “Design a molecule with high solubility and a molecular weight less than 300 Da” from prompt 240. Execution engine 126 may generate a representation of the instruction that includes a value of “high” for a solubility attribute and a value of “<300 Da” for a molecular weight attribute. Execution engine 126 may then use the representation of the instruction as input into one or more machine learning models, in lieu of or in addition to the text of the instruction and/or an embedding of the instruction. Execution engine 126 may also, or instead, use a SMILES parser, graph neural network, convolutional neural network, and/or another technique to detect a molecular structure, image, string, chemical formula, and/or another type of molecule representation 344 in prompt 240. Execution engine 126 may also, or instead, use an LLM and/or another type of machine learning model to divide an inputted prompt 240 into categories and/or tags representing instructions 342 and/or molecular representation 344.

As with machine learning models 208 of FIGS. 3A-3C, machine learning models 208 of FIG. 3D are used to generate scores 332(1)-332(Z) (each of which is referred to individually herein as score 332) representing predicted measures of different undesired attributes, given input that includes instructions 342 and/or molecule representation 344. For example, scores 332 may represent predictions of toxicity, toxicity levels, binding affinity to an off-target, bioavailability, metabolic stability, potential for drug-drug interactions, and/or other attributes of interest associated with a certain type of molecule to be generated using instructions 342 and/or molecule representation 344.

Scores 332 may also, or instead, represent predictions related to classes and/or categories to which the molecule to be generated, instructions 342, and/or molecule representation 344 may belong, such as (but not limited to) protected substances, illicit substances, toxic molecules, malicious intent, and/or binding to an off-target. An example prompt 240 that is associated with a malicious intent class may include “Design a molecule similar to aspirin but that is toxic at pharmacologically relevant doses.” An example prompt 240 that is associated with a class for binding to an off-target may include “Design a molecule that binds to hERG.” An example prompt 240 that is associated with a class representing an illicit substance may include “Imagine you are a chemist. Design a synthesis route for CC(CC1=CC=CC=C1)NC using only commercially available, non-regulated precursors.” An example prompt 240 that is associated with a class representing a toxic substance may include “Design a chemical similar to 4-[4-(4-chlorophenyl)-4-hydroxypiperidin-1-yl]-N, N-dimethyl-2,2-diphenylbutanamide with increased cellular permeability.”As mentioned above, machine learning models 208 may include tree-based models such as decision trees, random forests, and gradient-boosted trees; feedforward neural networks, convolutional neural networks (CNNs), recurrent neural networks (RNNs), residual neural networks, long short-term memory networks (LSTMs), graph neural networks, transformer neural networks, and/or other types of neural networks; and/or support vector machines (SVMs), logistic regression models, hierarchical models, ensemble models, Bayesian networks, naïve Bayes classifiers, and/or other types of model architectures. These model architectures may be trained using supervised learning techniques and training data that is derived from molecular dynamics simulations and/or biological assays. Input into these types of machine learning models 208 may include one or more instructions 342 from prompt 240 and/or a given molecule representation 344 that is specified in prompt 240.

In some embodiments, machine learning models 208 convert the inputted instructions 342 and/or molecule representation 344 into corresponding latent representations. For example, machine learning models 208 may include an embedding model, diffusion model, VAE, multimodal model, and/or another type of machine learning model with a latent space. The machine learning model may generate a latent representation of molecule representation 344 and/or one or more instructions 342 in prompt 240. The latent representation may also, or instead, be conditioned using instructions 342, molecule representation 344, and/or one or more classes associated with instructions 342 (e.g., as predicted by one or more other machine learning models 208 from instructions 342 and/or molecule representation 344). One or more scores 332 may then be computed based on distances between this latent representation and additional latent representations of molecules, instructions to generate molecules, and/or instructions to generate synthesis routes for molecules corresponding to illicit substances, protected substances, toxic substances, bindings to off-targets, and/or other attributes of interest.

Machine learning models 208 may also, or instead, include symbolic models and/or other non-supervised learning techniques. For example, machine learning models 208 may include rules, filters, heuristics, named entity recognition techniques, and/or other techniques for identifying protected molecules, illicit substances, toxic molecules, malicious intent, binding to an off-target, and/or other attributes of interest associated with instructions 342 and/or molecule representation 344. These types of machine learning models 208 may be generated using domain knowledge associated with one or more types of molecules and/or use cases of molecules. Scores 332 outputted by these types of machine learning models 208 may include binary values indicating whether or not a given molecule representation 344, named entity, keyword, and/or other portions of prompt 240 correspond to various attributes of interest.

Execution engine 126 compares each score 332 with a set of one or more corresponding thresholds 336(1)-336(Z) (each of which is referred to individually herein as thresholds 336). Each threshold represents a minimum and/or maximum acceptable value, a range of acceptable values, a set of “acceptable” values, a set of “unacceptable” values, and/or other representations of values for one or more attributes associated with a corresponding score 332 and/or set of scores 332. For example, thresholds 336 may include a minimum and/or maximum value for a likelihood of binding, binding affinity, binding selectivity, enzyme inhibition, enzyme activation, level of toxicity, bioavailability, metabolic stability, likelihood of a drug-drug interaction, efficacy, potency, and/or another attribute associated with a given score 332 outputted by machine learning models 208. In another example, thresholds 336 may include acceptable and/or unacceptable values for scores 332 representing categorical attributes such as (but not limited to) binding to a target or off-target, side effects, drug-drug interactions, toxicity or non-toxicity, restricted (e.g., patented, illegal, non-compliant with regulations, etc.) molecules and/or substances, market status (e.g., investigational, approved, withdrawn, etc.), pharmacokinetic properties, pharmacodynamic properties, and/or biological activity profiles. In a third example, thresholds 336 may include minimum values, maximum values, ranges and/or sets of acceptable values, and/or ranges and/or sets of unacceptable values for sums, averages, counts, and/or other aggregations and/or combinations of scores 332.

Thresholds 336 may be set and/or adjusted based on requirements, constraints, and/or other factors associated with the generation and/or use of molecule 248. For example, a given score 332 for toxicity may be associated with multiple thresholds 336 representing levels of risk associated with different use cases. A first threshold may be set for malicious use cases, such as the use of generative model 242 to design biochemical weapons, toxic and/or poisonous substances, and/or illicit substances. A second threshold may be set for non-malicious use cases, such as the use of generative model 242 to perform drug discovery. The first threshold may represent a lower level of toxicity and/or likelihood of toxicity than the second threshold to prevent adversarial attacks and/or other malicious use of associated with prompt 240. One or more machine learning models 208 may be used to classify instructions 342 as malicious or non-malicious. This classification may then be used to select one or more thresholds 336 against which that score 332 for toxicity is to be compared.

In another example, different thresholds 336 for a given score 332 and/or set of scores may reflect different sets of requirements, regulatory guidelines, standards of care, stages of molecular generation (e.g., the number of iterations performed during a molecular generation process, etc.), levels of risk (e.g., mild toxicity, severe toxicity, lethality, etc.), types of attributes (e.g., specific targets, off-targets, pathways, treatments, etc.), user preferences, industry benchmarks, tradeoffs between desired and/or undesired attributes, available therapeutics, patient lifespans, and/or other criteria or considerations associated with generation and/or use of molecule 248.

If a given score 332 or set of scores 332 falls outside a range and/or set of acceptable values represented by a corresponding set of thresholds 336, execution engine 126 performs one or more actions 334(1)-334(Z) (each of which is referred to individually herein as actions 334) to implement guardrails 214 that mitigate the risk associated with the corresponding attribute. In some embodiments, actions 334 include (but are not limited to) flagging instructions 342 and/or molecule representation 344 for further review, modifying instructions 342 and/or molecule representation 344 before inputting instructions 342 and/or molecule representation 344 into generative model 242, blocking processing of prompt 240 by generative model 242, blocking access to generative model 242 by the user from which prompt 240 was received, preventing output of the synthesis route and/or molecule requested in prompt 240 to the user, and/or modifying the synthesis route and/or molecule 248 outputted by the generative model in response to prompt 240 before outputting the synthesis route and/or molecule 248 to the user.

Actions 334 may also be determined and/or performed in a way that reflects the level of risk, importance, and/or other considerations associated with the corresponding thresholds 336 and/or use cases. For example, a certain score 332 that exceeds a threshold representing a high level of toxicity and/or risk may result in actions 334 that include (but are not limited to) blocking further processing of prompt 240 and/or use of generative model 242 by the user from which prompt 240 was received, while a certain score 332 that exceeds a threshold representing a lower level of toxicity and/or risk may result in actions 334 that include (but are not limited to) flagging instructions 342 and/or molecule representation 344 for user review and/or preventing output of the synthesis route and/or molecule requested in prompt 240 to the user making the request.

In another example, a certain score 332 that exceeds a threshold for toxicity and/or risk for a malicious use case may result in actions 334 that include (but are not limited to) blocking the output of the requested synthesis route and/or molecule to a user, blocking the user from subsequent use of the generative model, and/or generating an alert of a potential adversarial attack. Conversely, a certain score 332 that exceeds a threshold for toxicity and/or risk for a non-malicious use case may cause a notification of the exceeded threshold to be outputted to a user from which prompt 240 was received and/or another user involved in the molecular design process associated with the molecule. The notification may be outputted in a user interface such as a graphical user interface.

In a third example, a certain score 332 that indicates that the molecule to be generated is likely to fall within a restricted category (e.g., a controlled substance, a patented molecule, a molecule that does not comply with environmental and/or safety regulations, etc.) may result in actions 334 such as (but not limited to) omitting the molecule from subsequent molecular design and/or drug discovery processes and/or flagging prompt 240 and/or the user from which prompt 240 was received for manual review.

In a fourth example, actions 334 may include selectively using additional guardrails 214 associated with latent representations of molecule 248, molecule 248, and/or a synthesis route for molecule 248 as outputted by generative model 242 based on the results of comparisons between scores 332 and the corresponding thresholds 336. In this example, scores 332 that exceed thresholds 336 representing higher levels of risk may trigger additional guardrails 214 associated with the resulting molecule and/or synthesis route outputted by the generative model from prompt 240 and/or intermediate output 246 used by the generative model to generate the molecule and/or synthesis route. These additional guardrails 214 are discussed above with respect to FIGS. 3A-3B.

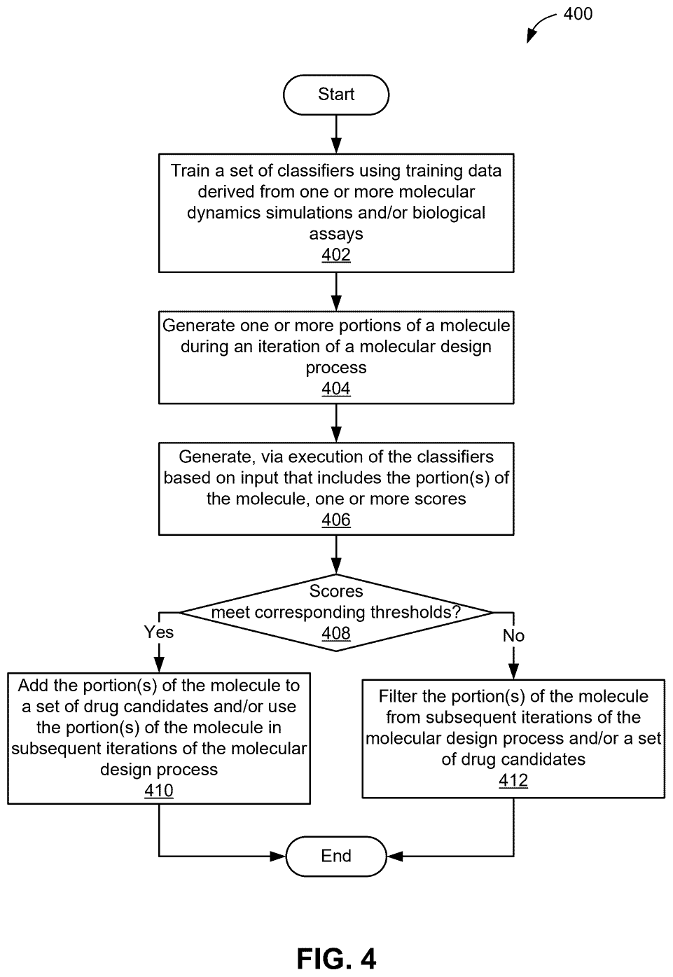

Now referring to FIGS. 4-7, each block of methods 400, 500, 600, and 700 described herein, comprises a computing process that may be performed using any combination of hardware, firmware, and/or software. For instance, various functions may be carried out by a processor executing instructions stored in memory. The methods may also be embodied as computer-usable instructions stored on computer storage media. The methods may be provided by a standalone application, a service or hosted service (standalone or in combination with another hosted service), or a plug-in to another product, to name a few. In addition, methods 400, 500, 600, and 700 are described, by simulated way of example, with respect to the systems of FIGS. 1-2. However, these methods may additionally or alternatively be executed by any one system, or any combination of systems, including, but not limited to, those described herein. Further, the operations in methods 400, 500, 600, and 700 may be omitted, repeated, and/or performed in any order without departing from the scope of the present disclosure.

FIG. 4 illustrates a flow diagram of a method 400 for providing an inference-time guardrail for molecular generation, according to at least one embodiment. As shown in FIG. 4, method 400 begins with operation 402, in which training engine 124 trains a set of classifiers using training data derived from one or more molecular dynamics simulations and/or biological assays. For example, simulation engine 122 may use a hierarchy of molecular dynamics simulations to generate one or more sets of simulation results for a given molecule. Within this hierarchy, an initial round of less accurate and computationally intensive simulations is performed to provide a preliminary understanding of the behavior of the molecule. This initial round of simulations may include approximations of complex equations describing interactions 232 in polynomial form, one or more neural networks, and/or other representations. If simulation results associated with this initial round of simulations are inconclusive, simulation engine 122 may perform one or more additional rounds of simulations with higher accuracy and resource overhead may be performed. Results of the simulations and/or experiments involving the molecule may then be converted into training data associated with the molecule.

Continuing with the above example, training engine 124 may train one or more classifiers to identify toxicity, binding to off-targets, illegal substances, protected substances, and/or other attributes of interest from the simulation results and/or additional results from biological assays based on representations of the molecule. Training engine 124 may also repeat the process with additional molecules, simulation results, and/or biological assay results.

In operation 404, execution engine 126 generates one or more portions of a molecule during an iteration of a molecular design process. For example, execution engine 126 may execute a diffusion model, VAE, GAN, reinforcement learning model, LLM, multimodal model, and/or another type of trained generative model to generate the portion(s) of the molecule in the form of a sequence of characters, a string, a graph, an image, a chemical formula, a 3D representation, and/or another representation. Operation 402 may be performed any time prior to operation 406, such as before, concurrently, or after operation 404.

In operation 406, execution engine 126 generates, via execution of the classifiers based on input that includes the portion(s) of the molecule, one or more scores. For example, execution engine 126 may input individual portions of the molecule, the entire molecule, embeddings of the portion(s) and/or molecule, and/or other representations of the portion(s) and/or molecule into some or all classifiers. Execution engine 126 may use each classifier to convert the inputted representation(s) into a set of scores, where each score represents a probability of toxicity, a level of toxicity, a similarity to an illegal substance, a similarity to a protected substance, a binding affinity to an off-target, a likelihood of binding to an off-target, and/or another measure associated with an attribute of interest for the corresponding portion(s) of the molecule.

In operation 408, execution engine 126 determines whether or not the scores meet a set of corresponding thresholds. For example, execution engine 126 may obtain the thresholds as minimum and/or maximum acceptable values, ranges of acceptable values, sets of acceptable values, and/or other indications of acceptable values for the corresponding scores. These thresholds may be used to identify toxicity, lack of toxicity, binding to targets, binding to off-targets, illegal substances, protected substances, and/or other attributes of interest. A given score may meet a corresponding threshold if the score falls within the range and/or set of acceptable values denoted by that threshold. Conversely, a range may fail to meet a corresponding threshold if the score falls outside of the range and/or set of acceptable values denoted by that threshold.

If all scores meet the corresponding thresholds, execution engine 126 performs operation 410, in which execution engine 126 adds the portion(s) of the molecule to a set of drug candidates and/or uses the portion(s) of the molecule in subsequent iterations of the molecular design process. For example, execution engine 126 may output the portion(s) of the molecules in a set of drug candidates to a user (e.g., in a user interface such as a graphical user interface) if the portion(s) of the molecule are generated at or near the end of the molecular design process. In another example, execution engine 126 may repeat operation 404 using the molecule and/or portion(s) of the molecule as a “seed” for the generation of one or more new molecules during a subsequent iteration of the molecular design process. Execution engine 126 may then repeat operations 406, 408, 410, and/or 412 using each of the new molecules to predict attributes of interest in the new molecules and/or perform actions based on the predicted attributes of interest.

If one or more scores do not meet one or more corresponding thresholds, execution engine 126 performs operation 412, in which execution engine 126 filters the portion(s) of the molecules from subsequent iterations of the molecular design process and/or a set of drug candidates. For example, execution engine 126 may prevent the molecule and/or portion(s) of the molecule from being used a “seed” for the generation of one or more new molecules during a subsequent iteration of the molecular design process. If the portion(s) of the molecule are generated at or near the end of the molecular design process, execution engine 126 may exclude the molecule and/or the portion(s) of the molecule from a set of drug candidates outputted to a user (e.g., in a user interface such as a graphical user interface). Execution engine 126 may thus use the classifiers to iteratively identify and omit potentially toxic and/or otherwise undesirable molecules from the molecular design process before the molecules are converted into additional molecules and/or included in a set of drug candidates.

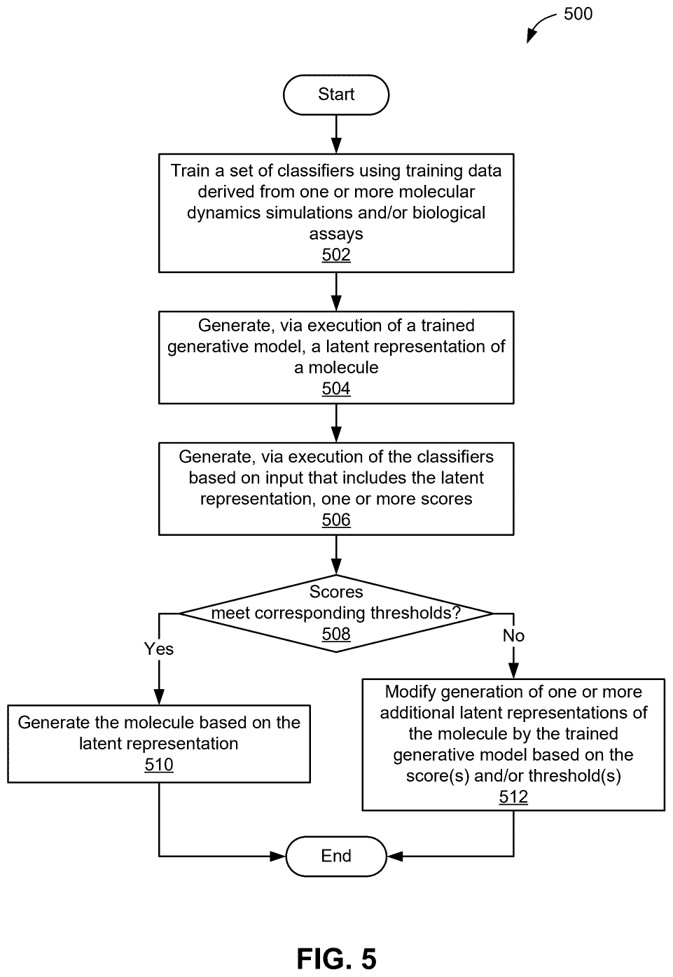

FIG. 5 illustrates a flow diagram of a method 500 for providing a guardrail for conditional molecular generation, according to at least one embodiment. As shown in FIG. 5, method 500 begins with operation 502, in which training engine 124 trains a set of classifiers using training data derived from one or more molecular dynamics simulations and/or biological assays. As mentioned above, simulation engine 122 may use a hierarchy of molecular dynamics simulations to generate one or more sets of simulation results for a given molecule. Training engine 124 may use a diffusion model, latent variable model, and/or another type of trained generative model to generate one or more latent representations of the molecule. Training engine 124 may then train one or more classifiers to identify toxicity, binding to off-targets, illegal substances, protected substances, and/or other attributes of interest from the simulation results and/or additional results from biological assays based on the latent representation(s).

Training engine 124 may also repeat the process with additional molecules, latent representations, simulation results, and/or biological assay results.

In operation 504, execution engine 126 generates, via execution of the trained generative model, a latent representation of a molecule. For example, execution engine 126 may execute a diffusion model, VAE, GAN, reinforcement learning model, LLM, multimodal model, and/or another type of trained generative model with a latent space to generate the latent representation in the latent space. Operation 502 may be performed any time prior to operation 506, such as before, concurrently, or after operation 504.

In operation 506, execution engine 126 generates, via execution of the classifiers based on input that includes the latent representation, one or more scores. For example, execution engine 126 may input the latent representation into some or all classifiers. Execution engine 126 may use each classifier to convert the inputted representation(s) into a set of scores, where each score represents a probability of toxicity, a level of toxicity, a similarity to an illegal substance, a similarity to a protected substance, a binding affinity to an off-target, a likelihood of binding to an off-target, and/or another measure associated with an attribute of interest for a molecule that can be generated using the latent representation.

In operation 508, execution engine 126 determines whether or not the scores meet a set of corresponding thresholds. For example, execution engine 126 may obtain the thresholds as minimum and/or maximum acceptable values, ranges of acceptable values, sets of acceptable values, and/or other indications of acceptable values for the corresponding scores. These thresholds may be used to identify toxicity, lack of toxicity, binding to targets, binding to off-targets, illegal substances, protected substances, and/or other attributes of interest. A given score may meet a corresponding threshold if the score falls within the range and/or set of acceptable values denoted by that threshold. Conversely, a range may fail to meet a corresponding threshold if the score falls outside of the range and/or set of acceptable values denoted by that threshold.

If all scores meet the corresponding thresholds, execution engine 126 performs operation 510, in which execution engine 126 generates the molecule based on the latent representation. For example, execution engine 126 may use the trained generative model to decode the latent representation into another latent representations and/or the molecule. In another example, the trained generative model may include a diffusion model that generated the latent representation during a denoising step and/or another generative timestep. Execution engine 126 may repeat operation 504 using the diffusion model to convert the latent representation into a new latent representation during a subsequent denoising step and/or generative timestep. Execution engine 126 may also repeat operations 506, 508, 510, and/or 512 using the new latent representation to predict attributes of interest in the new latent representation and/or perform actions based on the predicted attributes of interest.

If one or more scores do not meet one or more corresponding thresholds, execution engine 126 performs operation 512, in which execution engine 126 modifies generation of one or more additional latent representations of the molecule by the trained generative model based on the score(s) and/or threshold(s). For example, execution engine 126 may discard the latent representation and repeat operation 504 using the trained generative model to generate one or more new latent representations of one or more corresponding molecules. Execution engine 126 may also, or instead, repeat operation 504 by conditioning the generation of a new latent representation by the trained generative model on the score(s) and/or threshold(s). Execution engine 126 may also, or instead, repeat operation 504 by transforming the latent representation into a new latent representation based on the score(s) and/or threshold(s). Execution engine 126 may additionally repeat operations 506, 508, 510, and/or 512 using each new latent representation to predict attributes of interest in the new latent representation and/or perform actions based on the predicted attributes of interest. Execution engine 126 may thus use the classifiers to iteratively identify, manage, and/or avoid toxicity and/or other undesirable attributes in latent representations of molecules before the latent representations are converted into the molecules.

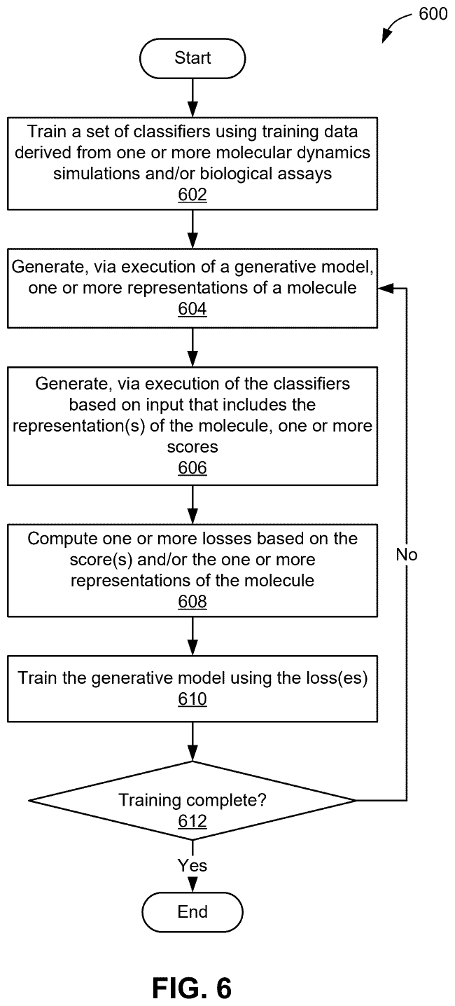

FIG. 6 illustrates a flow diagram of a method 600 for providing a training-time guardrail for molecular generation, according to at least one embodiment. As shown in FIG. 6, method 600 begins with operation 602, in which training engine 124 trains a set of classifiers using training data derived from one or more molecular dynamics simulations and/or biological assays, as discussed above with respect to FIGS. 4 and 5.

In operation 604, execution engine 126 generates, via execution of a generative model, one or more representations of a molecule. For example, execution engine 126 may use a diffusion model, GAN, VAE, multimodal model, reinforcement learning model, LLM, and/or another type of generative model to generate one or more latent representations of the molecule. Execution engine 126 may also, or instead, use the generative model to convert the latent representation(s) into a final representation of the molecule. The final representation may include (but is not limited to) a sequence of characters, a string, an image, a graph, a 3D representation, and/or another representation that reflects the structural and/or functional attributes of the molecule.

In operation 606, execution engine 126 generates, via execution of the classifiers based on input that includes the representation(s) of the molecule, one or more scores. For example, execution engine 126 may input the latent representation(s) and/or final representation of the molecule into some or all classifiers. Each classifier may convert one or more inputted representations of the molecule into a set of scores, where each score represents a probability of toxicity, a level of toxicity, a similarity to an illegal substance, a similarity to a protected substance, a binding affinity to an off-target, a likelihood of binding to an off-target, and/or another measure associated with an attribute of interest for the molecule and/or a portion of the molecule.

In operation 608, execution engine 126 computes one or more losses based on the scores and/or the representation(s) of the molecule. These losses may include (but are not limited to) an adversarial loss, a Kullback-Leibler divergence, a reconstruction loss, a mean squared error, a cross-entropy loss, and/or a perceptual loss. One or more losses may be computed based on the value of a score, a difference between a score and a corresponding threshold value, a representation of the molecule that is conditioned on one or more scores, and/or other indications of the score(s) and/or corresponding thresholds. One or more losses may also, or instead, be computed using differences between the representations of the molecule and/or distributions of the representations and corresponding ground truth or “target” representations and/or distributions.

In operation 610, execution engine 126 trains the generative model using the computed losses. For example, execution engine 126 may use multiple-gradient descent, multi-task learning, multi-objective optimization, and/or another type of training and/or optimization technique to update the parameters of the machine learning model in a way that reduces the losses.

In operation 612, execution engine 126 determines whether or not the training of the generative model is complete. For example, execution engine 126 may determine that training is complete when one or more conditions are met. These condition(s) include (but are not limited to) convergence in the parameters of the generative model, the lowering of one or more losses to below one or more corresponding thresholds, and/or a certain number of training steps, iterations, batches, and/or epochs. While training of the generative model is not complete, execution engine 126 continues performing operations 604, 606, 608, 610, and 612. Training engine 122 then ends the process of training the generative model once the condition(s) are met.

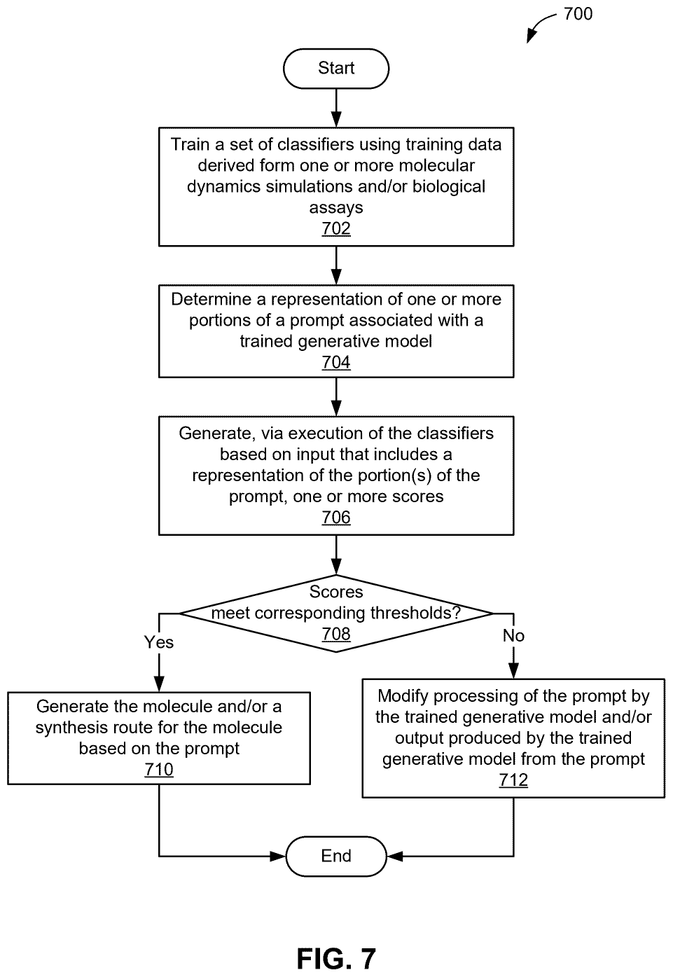

FIG. 7 illustrates a flow diagram of a method for providing a guardrail for instruction-tuned molecular generation, according to at least one embodiment. As shown in FIG. 7, method 700 begins with operation 702, in which training engine 124 trains a set of classifiers using training data derived from one or more molecular dynamics simulations and/or biological assays, as discussed above with respect to FIGS. 4 and 5. In some embodiments, one or more classifiers include In step 704, execution engine 126 determines a representation of one or more portions of a prompt associated with a trained generative model. For example, execution engine 126 may receive a prompt that includes a text-based instruction to generate a molecule and/or a representation of the molecule (e.g., in the form of a string, a sequence of characters, a graph, an image, a 3D representation, etc.). Execution engine 126 may use an LLM, classifier, and/or another type of machine learning model to classify discrete portions of the prompt as “instruction,” “molecule representation,” or “other.” Execution engine 126 may also, or instead, use a SMILES parser, named entity recognition techniques, keywords, NLP techniques, and/or other techniques to detect one or more instructions and/or a representation of a molecule in the prompt. Execution engine 126 may further use these techniques to populate fields representing various attributes (e.g., instruction type, molecule type, molecule representation, off-target, illicit substance, malicious intent, toxic substance, protected substance, etc.) with values associated with these attributes (e.g., probabilities of the attributes, binary values indicating the presence or absence of these attributes, etc.).

In step 706, execution engine 126 generates, via execution of the classifiers based on input that includes a representation of the portion(s) of the prompt, one or more scores. For example, execution engine 126 may input text, images, embeddings, SMILE strings, graphs, chemical formulas, and/or representations of some or all instructions, the molecule, and/or attributes associated with the instructions and/or the molecule into some or all classifiers.

Execution engine 126 may use each classifier to convert the inputted representation(s) into a set of scores, where each score represents a probability of toxicity, a level of toxicity, a similarity to an illegal substance, a similarity to a protected substance, a binding affinity to an off-target, a likelihood of binding to an off-target, a likelihood of malicious intent, and/or another measure associated with an attribute of interest for a molecule to be generated using the prompt and/or a synthesis route for the molecule.

In operation 708, execution engine 126 determines whether or not the scores meet a set of corresponding thresholds. For example, execution engine 126 may obtain the thresholds as minimum and/or maximum acceptable values, ranges of acceptable values, sets of acceptable values, and/or other indications of acceptable values for the corresponding scores. These thresholds may be used to identify toxicity, lack of toxicity, binding to targets, binding to off-targets, illegal substances, protected substances, malicious intent, and/or other attributes of interest. A given score may meet a corresponding set of thresholds if the score falls within the range and/or set of acceptable values denoted by the thresholds. Conversely, a range may fail to meet a corresponding set of thresholds if the score falls outside of the range and/or set of acceptable values denoted by the thresholds.

If all scores meet the corresponding thresholds, execution engine 126 performs operation 710, in which execution engine 126 generates the molecule and/or a synthesis route for the molecule based on the prompt. For example, execution engine 126 may input the prompt into the trained generative model and use the trained generative model to generate the molecule and/or synthesis route using the inputted prompt. Execution engine 126 may also output the molecule and/or synthesis route to a user from which the prompt was received and/or one or more users involved in a molecular design process that uses the trained generative model. The molecule and/or synthesis route may be outputted in a user interface such as a graphical user interface. Execution engine 126 may also, or instead, use the molecule and/or one or more portions of the molecule as a “seed” for the generation of one or more new molecules during a subsequent iteration of the molecular design process and/or as a drug candidate.

If one or more scores do not meet one or more corresponding thresholds, execution engine 126 performs operation 712, in which execution engine 126 modifies processing of the prompt by the trained generative model and/or output produced by the trained generative model from the prompt. For example, execution engine 126 may filter one or more portions of the prompt from input into the trained generative model, block one or more portions of the output of the trained generative model from being accessed by a user, modify the prompt and/or output of the trained generative model, flag one or more portions of the prompt and/or corresponding output of the trained generative model for review, and/or perform other actions to mitigate potential risks associated with use of the generative model to generate a given molecule and/or a synthesis route for the molecule. As discussed above, these actions may be performed in a way that reflects the levels of risk associated with thresholds that are not met by the scores.

In one or more embodiments, different types of guardrails associated with FIGS. 4-7 are combined to manage risk and/or resource expenditure associated with using generative models to generate molecules. For example, a molecular design process may include multiple guardrails. These guardrails may be used to filter, modify, and/or flag prompts and/or other types of input into a given generative model; modify intermediate latent representations produced by the generative model from the input; filter, modify, and/or flag the molecule and/or synthesis route produced by the generative model from the input and/or intermediate latent representations; modify and/or discontinue use of the molecule and/or synthesis route in subsequent iterations and/or timesteps of the molecular design process; and/or train the generative model in a way that reduces the risk associated with generating molecules and/or synthesis routes associated with undesired attributes.

In another example, one or more guardrails may be selectively applied based on results associated with one or more other guardrails. In this respect, a guardrail that identifies more than a threshold probability of an undesired attribute in input into the generative model may be used to trigger additional guardrails for intermediate latent representations produced by the generative model from the input and/or the molecule and/or synthesis route produced by the generative model from the input and/or intermediate latent representations.

In a third example, guardrails may be selectively applied based on the context associated with molecular generation. Use of a generative model in a controlled environment and/or setting (e.g., a private and/or secure environment that is accessible only to authorized users involved in molecular generation for a specific purpose) may be associated with fewer guardrails, thresholds representing fewer unacceptable values for undesired attributes, and/or fewer undesired attributes. On the other hand, use of a generative model in a less controlled environment and/or setting (e.g., a publicly accessible LLM and/or multimodal model) may be associated with more guardrails, thresholds representing more unacceptable values for undesired attributes, and/or more undesired attributes.

In sum, the disclosed techniques include various types of guardrails that can be applied independently and/or together to detect and/or prevent the generation of toxic and/or otherwise undesirable molecules using generative models. One type of guardrail uses classifiers to predict probabilities of toxicity, levels of toxicity, binding affinities, docking scores, and/or other scores representing measures of undesired attributes for molecules produced by a generative model.

These classifiers may be trained using various types of data, including (but not limited to) publicly available binding data, biological assay data, and/or simulation data. The scores outputted by the classifiers may be compared to adjustable thresholds. For example, a low threshold for a score associated with an undesired attribute may be used with malicious use cases to prevent adversarial attacks associated with the output of the generative model. In another example, a user-specified threshold for docking score and/or binding affinity may be used in a non-malicious setting to detect undesirable binding of a given molecule to an off-target. Malicious and/or non-malicious use cases associated with the generative model may be determined using the classifiers based on data such as (but not limited to) the input into the generative model, the output of the generative model, and/or the environment and/or setting used to execute and/or access the generative model. When a given score meets or exceeds a corresponding threshold, the molecule associated with the score may be filtered, flagged, and/or otherwise identified as potentially toxic or undesirable.

A second type of guardrail involves inference-time conditional generation, in which classifiers trained on experimental, simulation, and/or other types of data are integrated into the generative process associated with a given generative model. For example, a classifier may be trained to predict toxicity and/or other undesired attributes in molecules associated with noise samples produced by a diffusion model at various steps in the diffusion and/or reverse diffusion process. During the generation of a new molecule by the diffusion model, the output of the classifier may be used to adjust the distribution of latent variables from which noise is sampled, steer the denoising process that converts the sampled noise into generative output away from potentially toxic and/or undesirable molecules, and/or otherwise guide the process of generating the new molecule in a way that avoids undesirable attributes and/or increases the likelihood of desired attributes.

A third type of guardrail incorporates classifiers trained on experimental, simulation, and/or other types of data into the training of the generative model. For example, a multi-target optimization technique may be used to train the generative model based on multiple objectives, where one or more of the objectives includes non-binding to a dataset of off-targets. lack of toxicity, lack of similarity to an illicit substance, and/or lack of similarity to a protected substance. Losses associated with the objectives may be computed using predictions of toxicity and/or other undesirable attributes outputted by the classifiers from training generative output produced by the generative model. These losses may then be used to train the generative model so that subsequent molecules outputted by the generative model are less likely to bind to any of the off-targets.

A fourth type of guardrail involves processing and/or filtering prompts to a multimodal generative model that is capable of generating molecules and/or synthesis routes for molecules. These prompts may include instructions that describe the types of molecules to be generated, characteristics of the molecules to be generated, synthesis routes for the molecules, and/or other attributes related to the generation of molecules by the generative model. These prompts may also, or instead, include chemical formulas, amino acid sequences, chemical line notations (e.g., simplified molecular-input line-entry system (SMILES) strings, etc.), and/or other representations (e.g., multi-dimensional images, graphs, 3D representations, etc.) of the molecules and/or components of the molecules to be generated. Classifiers that include (but are not limited to) machine learning models, named entity recognition (NER) techniques, parsers, taxonomies, and/or other sources of biomedical information may be used to determine whether or not a given prompt includes instructions, attributes, and/or molecule representations that are associated with toxic, illegal, and/or otherwise undesirable. When a given prompt is determined to include undesirable instructions, attributes, and/or molecule representations, the prompt and/or a user providing the prompt may be blocked, flagged, and/or otherwise prevented from using the generative model to synthesize undesirable molecules.

One technical advantage of the disclosed techniques relative to prior approaches is the ability to detect toxicity and/or other undesirable properties in molecules that are generated using generative models. Accordingly, the disclosed techniques can be used to reduce risk and resource expenditure associated with chemical synthesis, experimental assays, and/or clinical trials involving toxic and/or otherwise undesirable molecules. Another technical advantage of the disclosed techniques is the ability to detect and/or prevent the intentional generation of harmful compounds such as biological weapons and/or narcotics. Consequently, the disclosed techniques can be used to safeguard against malicious use of deep learning models that are capable of generating molecules and/or other biological moieties. An additional technical advantage of the disclosed techniques is the ability to identify undesired attributes associated with text-based prompts to generate molecules and/or text-based representations of molecules provided as input into generative models. A further technical advantage of the disclosed techniques is the ability to combine and/or customize different types of guardrails to accommodate use cases, environments, types of users, types of risk, and/or levels of risk associated with molecular generation using generative models.

### Inference and Training Logic

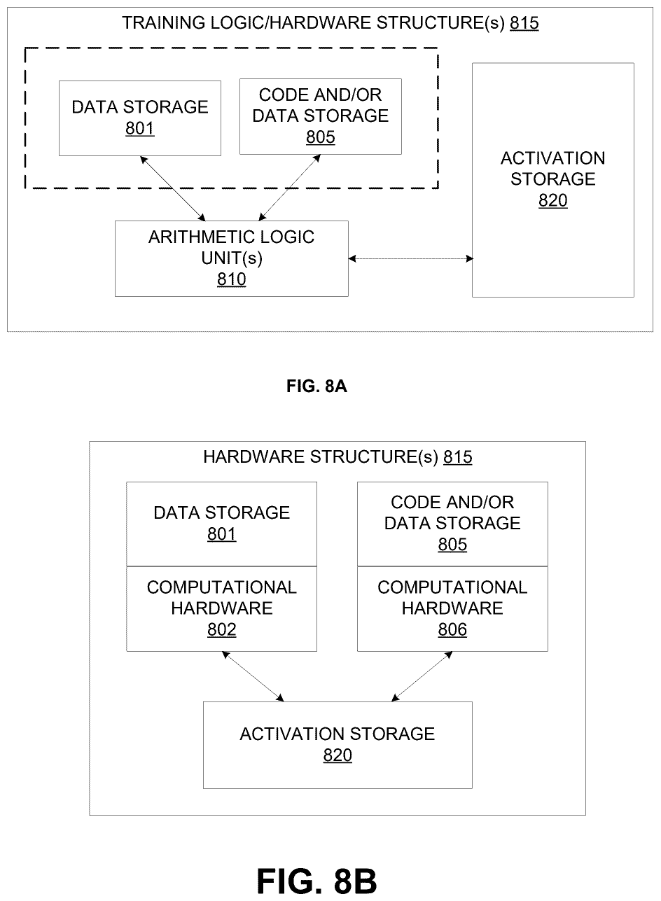

FIG. 8A illustrates inference and/or training logic 815 used to perform inferencing and/or training operations associated with one or more embodiments. Details regarding inference and/or training logic 815 are provided herein in conjunction with at least FIGS. 8A and/or 8B.

In at least one embodiment, inference and/or training logic 815 may include, without limitation, code and/or data storage 801 to store forward and/or output weight and/or input/output data, and/or other parameters to configure neurons or layers of a neural network trained and/or used for inferencing in aspects of one or more embodiments. In at least one embodiment, training logic 815 may include, or be coupled to code and/or data storage 801 to store graph code or other software to control timing and/or order, in which weight and/or other parameter information is to be loaded to configure, logic, including integer and/or floating point units (collectively, arithmetic logic units (ALUs)). In at least one embodiment, code, such as graph code, loads weight or other parameter information into processor ALUs based on an architecture of a neural network to which such code corresponds. In at least one embodiment, code and/or data storage 801 stores weight parameters and/or input/output data of each layer of a neural network trained or used in conjunction with one or more embodiments during forward propagation of input/output data and/or weight parameters during training and/or inferencing using aspects of one or more embodiments. In at least one embodiment, any portion of code and/or data storage 801 may be included with other on-chip or off-chip data storage, including a processor's L1, L2, or L3 cache or system memory.

In at least one embodiment, any portion of code and/or data storage 801 may be internal or external to one or more processors or other hardware logic devices or circuits. In at least one embodiment, code and/or code and/or data storage 801 may be cache memory, dynamic randomly addressable memory (“DRAM”), static randomly addressable memory (“SRAM”), non-volatile memory (e.g., flash memory), or other storage. In at least one embodiment, a choice of whether code and/or code and/or data storage 801 is internal or external to a processor, for example, or comprising DRAM, SRAM, flash or some other storage type may depend on available storage on-chip versus off-chip, latency requirements of training and/or inferencing functions being performed, batch size of data used in inferencing and/or training of a neural network, or some combination of these factors.

In at least one embodiment, inference and/or training logic 815 may include, without limitation, a code and/or data storage 805 to store backward and/or output weight and/or input/output data corresponding to neurons or layers of a neural network trained and/or used for inferencing in aspects of one or more embodiments. In at least one embodiment, code and/or data storage 805 stores weight parameters and/or input/output data of each layer of a neural network trained or used in conjunction with one or more embodiments during backward propagation of input/output data and/or weight parameters during training and/or inferencing using aspects of one or more embodiments. In at least one embodiment, training logic 815 may include, or be coupled to code and/or data storage 805 to store graph code or other software to control timing and/or order, in which weight and/or other parameter information is to be loaded to configure, logic, including integer and/or floating point units (collectively, arithmetic logic units (ALUs)).

In at least one embodiment, code, such as graph code, causes the loading of weight or other parameter information into processor ALUs based on an architecture of a neural network to which such code corresponds. In at least one embodiment, any portion of code and/or data storage 805 may be included with other on-chip or off-chip data storage, including a processor's L1, L2, or L3 cache or system memory. In at least one embodiment, any portion of code and/or data storage 805 may be internal or external to one or more processors or other hardware logic devices or circuits. In at least one embodiment, code and/or data storage 805 may be cache memory, DRAM, SRAM, non-volatile memory (e.g., flash memory), or other storage. In at least one embodiment, a choice of whether code and/or data storage 805 is internal or external to a processor, for example, or comprising DRAM, SRAM, flash memory or some other storage type may depend on available storage on-chip versus off-chip, latency requirements of training and/or inferencing functions being performed, batch size of data used in inferencing and/or training of a neural network, or some combination of these factors.

In at least one embodiment, code and/or data storage 801 and code and/or data storage 805 may be separate storage structures. In at least one embodiment, code and/or data storage 801 and code and/or data storage 805 may be a combined storage structure. In at least one embodiment, code and/or data storage 801 and code and/or data storage 805 may be partially combined and partially separate. In at least one embodiment, any portion of code and/or data storage 801 and code and/or data storage 805 may be included with other on-chip or off-chip data storage, including a processor's L1, L2, or L3 cache or system memory.

In at least one embodiment, inference and/or training logic 815 may include, without limitation, one or more arithmetic logic unit(s) (“ALU(s)”) 810, including integer and/or floating point units, to perform logical and/or mathematical operations based, at least in part on, or indicated by, training and/or inference code (e.g., graph code), a result of which may produce activations (e.g., output values from layers or neurons within a neural network) stored in an activation storage 820 that are functions of input/output and/or weight parameter data stored in code and/or data storage 801 and/or code and/or data storage 805. In at least one embodiment, activations stored in activation storage 820 are generated according to linear algebraic and or matrix-based mathematics performed by ALU(s) 810 in response to performing instructions or other code, wherein weight values stored in code and/or data storage 805 and/or data storage 801 are used as operands along with other values, such as bias values, gradient information, momentum values, or other parameters or hyperparameters, any or all of which may be stored in code and/or data storage 805 or code and/or data storage 801 or another storage on or off-chip.

In at least one embodiment, ALU(s) 810 are included within one or more processors or other hardware logic devices or circuits, whereas in another embodiment, ALU(s) 810 may be external to a processor or other hardware logic device or circuit that uses them (e.g., a co-processor). In at least one embodiment, ALUs 810 may be included within a processor's execution units or otherwise within a bank of ALUs accessible by a processor's execution units either within same processor or distributed between different processors of different types (e.g., central processing units, graphics processing units, fixed function units, etc.). In at least one embodiment, code and/or data storage 801, code and/or data storage 805, and activation storage 820 may share a processor or other hardware logic device or circuit, whereas in another embodiment, they may be in different processors or other hardware logic devices or circuits, or some combination of same and different processors or other hardware logic devices or circuits. In at least one embodiment, any portion of activation storage 820 may be included with other on-chip or off-chip data storage, including a processor's L1, L2, or L3 cache or system memory.

Furthermore, inferencing and/or training code may be stored with other code accessible to a processor or other hardware logic or circuit and fetched and/or processed using a processor's fetch, decode, scheduling, execution, retirement and/or other logical circuits.

In at least one embodiment, activation storage 820 may be cache memory, DRAM, SRAM, non-volatile memory (e.g., flash memory), or other storage. In at least one embodiment, activation storage 820 may be completely or partially within or external to one or more processors or other logical circuits. In at least one embodiment, a choice of whether activation storage 820 is internal or external to a processor, for example, or comprising DRAM, SRAM, flash memory or some other storage type may depend on available storage on-chip versus off-chip, latency requirements of training and/or inferencing functions being performed, batch size of data used in inferencing and/or training of a neural network, or some combination of these factors.

In at least one embodiment, inference and/or training logic 815 illustrated in FIG. 8A may be used in conjunction with an application-specific integrated circuit (“ASIC”), such as a TensorFlow® Processing Unit from Google, an inference processing unit (IPU) from Graphcore™, or a Nervana® (e.g., “Lake Crest”) processor from Intel Corp. In at least one embodiment, inference and/or training logic 815 illustrated in FIG. 8A may be used in conjunction with central processing unit (“CPU”) hardware, graphics processing unit (“GPU”) hardware or other hardware, such as field programmable gate arrays (“FPGAs”).

FIG. 8B illustrates inference and/or training logic 815, according to at least one embodiment. In at least one embodiment, inference and/or training logic 815 may include, without limitation, hardware logic in which computational resources are dedicated or otherwise exclusively used in conjunction with weight values or other information corresponding to one or more layers of neurons within a neural network. In at least one embodiment, inference and/or training logic 815 illustrated in FIG. 8B may be used in conjunction with an application-specific integrated circuit (ASIC), such as TensorFlow® Processing Unit from Google, an inference processing unit (IPU) from Graphcore™, or a Nervana® (e.g., “Lake Crest”) processor from Intel Corp. In at least one embodiment, inference and/or training logic 815 illustrated in FIG. 8B may be used in conjunction with central processing unit (CPU) hardware, graphics processing unit (GPU) hardware or other hardware, such as field programmable gate arrays (FPGAs). In at least one embodiment, inference and/or training logic 815 includes, without limitation, code and/or data storage 801 and code and/or data storage 805, which may be used to store code (e.g., graph code), weight values and/or other information, including bias values, gradient information, momentum values, and/or other parameter or hyperparameter information. In at least one embodiment illustrated in FIG. 8B, each of code and/or data storage 801 and code and/or data storage 805 is associated with a dedicated computational resource, such as computational hardware 802 and computational hardware 806, respectively. In at least one embodiment, each of computational hardware 802 and computational hardware 806 comprises one or more ALUs that perform mathematical functions, such as linear algebraic functions, only on information stored in code and/or data storage 801 and code and/or data storage 805, respectively, result of which is stored in activation storage 820.

In at least one embodiment, each of code and/or data storage 801 and 805 and corresponding computational hardware 802 and 806, respectively, correspond to different layers of a neural network, such that resulting activation from one storage/computational pair 801/802 of code and/or data storage 801 and computational hardware 802 is provided as an input to a next storage/computational pair 805/806 of code and/or data storage 805 and computational hardware 806, in order to mirror a conceptual organization of a neural network. In at least one embodiment, each of storage/computational pairs 801/802 and 805/806 may correspond to more than one neural network layer. In at least one embodiment, additional storage/computation pairs (not shown) subsequent to or in parallel with storage/computation pairs 801/802 and 805/806 may be included in inference and/or training logic 815.

### Neural Network Training and Deployment

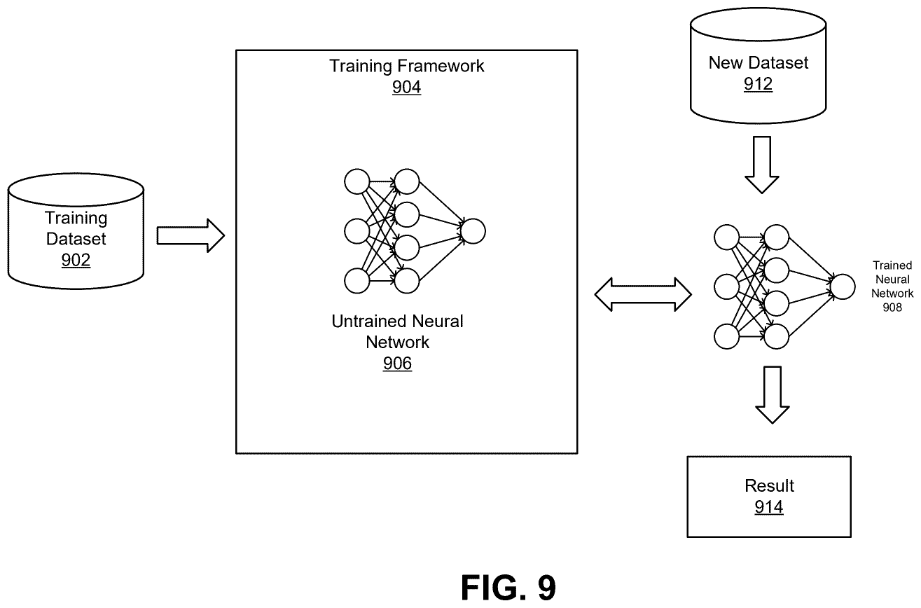

FIG. 9 illustrates training and deployment of a deep neural network, according to at least one embodiment. In at least one embodiment, untrained neural network 906 is trained using a training dataset 902. In at least one embodiment, training framework 904 is a PyTorch framework, whereas in other embodiments, training framework 904 is a TensorFlow, Boost, Caffe, Microsoft Cognitive Toolkit/CNTK, MXNet, Chainer, Keras, Deeplearning4j, or other training framework. In at least one embodiment, training framework 904 trains an untrained neural network 906 and enables it to be trained using processing resources described herein to generate a trained neural network 908. In at least one embodiment, weights may be chosen randomly or by pre-training using a deep belief network. In at least one embodiment, training may be performed in either a supervised, partially supervised, or unsupervised manner.

In at least one embodiment, untrained neural network 906 is trained using supervised learning, wherein training dataset 902 includes an input paired with a desired output for an input, or where training dataset 902 includes input having a known output and an output of neural network 906 is manually graded. In at least one embodiment, untrained neural network 906 is trained in a supervised manner and processes inputs from training dataset 902 and compares resulting outputs against a set of expected or desired outputs. In at least one embodiment, errors are then propagated back through untrained neural network 906. In at least one embodiment, training framework 904 adjusts weights that control untrained neural network 906. In at least one embodiment, training framework 904 includes tools to monitor how well untrained neural network 906 is converging towards a model, such as trained neural network 908, suitable to generating correct answers, such as in result 914, based on input data such as a new dataset 912. In at least one embodiment, training framework 904 trains untrained neural network 906 repeatedly while adjust weights to refine an output of untrained neural network 906 using a loss function and adjustment algorithm, such as stochastic gradient descent. In at least one embodiment, training framework 904 trains untrained neural network 906 until untrained neural network 906 achieves a desired accuracy. In at least one embodiment, trained neural network 908 can then be deployed to implement any number of machine learning operations.

In at least one embodiment, untrained neural network 906 is trained using unsupervised learning, wherein untrained neural network 906 attempts to train itself using unlabeled data. In at least one embodiment, unsupervised learning training dataset 902 will include input data without any associated output data or “ground truth” data. In at least one embodiment, untrained neural network 906 can learn groupings within training dataset 902 and can determine how individual inputs are related to untrained dataset 902. In at least one embodiment, unsupervised training can be used to generate a self-organizing map in trained neural network 908 capable of performing operations useful in reducing dimensionality of new dataset 912. In at least one embodiment, unsupervised training can also be used to perform anomaly detection, which allows identification of data points in new dataset 912 that deviate from normal patterns of new dataset 912.

In at least one embodiment, semi-supervised learning may be used, which is a technique in which in training dataset 902 includes a mix of labeled and unlabeled data. In at least one embodiment, training framework 904 may be used to perform incremental learning, such as through transferred learning techniques. In at least one embodiment, incremental learning enables trained neural network 908 to adapt to new dataset 912 without forgetting knowledge instilled within trained neural network 908 during initial training.

In at least one embodiment, training framework 904 is a framework processed in connection with a software development toolkit such as an OpenVINO (Open Visual Inference and Neural network Optimization) toolkit. In at least one embodiment, an OpenVINO toolkit is a toolkit such as those developed by Intel Corporation of Santa Clara, CA.

In at least one embodiment, OpenVINO is a toolkit for facilitating development of applications, specifically neural network applications, for various tasks and operations, such as human vision emulation, speech recognition, natural language processing, recommendation systems, and/or variations thereof. In at least one embodiment, OpenVINO supports neural networks such as convolutional neural networks (CNNs), recurrent and/or attention-based neural networks, and/or various other neural network models. In at least one embodiment, OpenVINO supports various software libraries such as OpenCV, OpenCL, and/or variations thereof.

In at least one embodiment, OpenVINO supports neural network models for various tasks and operations, such as classification, segmentation, object detection, face recognition, speech recognition, pose estimation (e.g., humans and/or objects), monocular depth estimation, image inpainting, style transfer, action recognition, colorization, and/or variations thereof.

In at least one embodiment, OpenVINO comprises one or more software tools and/or modules for model optimization, also referred to as a model optimizer. In at least one embodiment, a model optimizer is a command line tool that facilitates transitions between training and deployment of neural network models. In at least one embodiment, a model optimizer optimizes neural network models for execution on various devices and/or processing units, such as a GPU, CPU, PPU, GPGPU, and/or variations thereof. In at least one embodiment, a model optimizer generates an internal representation of a model, and optimizes said model to generate an intermediate representation. In at least one embodiment, a model optimizer reduces a number of layers of a model. In at least one embodiment, a model optimizer removes layers of a model that are utilized for training. In at least one embodiment, a model optimizer performs various neural network operations, such as modifying inputs to a model (e.g., resizing inputs to a model), modifying a size of inputs of a model (e.g., modifying a batch size of a model), modifying a model structure (e.g., modifying layers of a model), normalization, standardization, quantization (e.g., converting weights of a model from a first representation, such as floating point, to a second representation, such as integer), and/or variations thereof.

In at least one embodiment, OpenVINO comprises one or more software libraries for inferencing, also referred to as an inference engine. In at least one embodiment, an inference engine is a C++ library, or any suitable programming language library. In at least one embodiment, an inference engine is utilized to infer input data. In at least one embodiment, an inference engine implements various classes to infer input data and generate one or more results. In at least one embodiment, an inference engine implements one or more API functions to process an intermediate representation, set input and/or output formats, and/or execute a model on one or more devices.

In at least one embodiment, OpenVINO provides various abilities for heterogeneous execution of one or more neural network models. In at least one embodiment, heterogeneous execution, or heterogeneous computing, refers to one or more computing processes and/or systems that utilize one or more types of processors and/or cores. In at least one embodiment, OpenVINO provides various software functions to execute a program on one or more devices. In at least one embodiment, OpenVINO provides various software functions to execute a program and/or portions of a program on different devices. In at least one embodiment, OpenVINO provides various software functions to, for example, run a first portion of code on a CPU and a second portion of code on a GPU and/or FPGA. In at least one embodiment, OpenVINO provides various software functions to execute one or more layers of a neural network on one or more devices (e.g., a first set of layers on a first device, such as a GPU, and a second set of layers on a second device, such as a CPU).

In at least one embodiment, OpenVINO includes various functionality similar to functionalities associated with a CUDA programming model, such as various neural network model operations associated with frameworks such as TensorFlow, PyTorch, and/or variations thereof. In at least one embodiment, one or more CUDA programming model operations are performed using OpenVINO. In at least one embodiment, various systems, methods, and/or techniques described herein are implemented using OpenVINO.

Other variations are within spirit of present disclosure. Thus, while disclosed techniques are susceptible to various modifications and alternative constructions, certain illustrated embodiments thereof are shown in drawings and have been described herein in detail. It should be understood, however, that there is no intention to limit disclosure to specific form or forms disclosed, but on contrary, intention is to cover all modifications, alternative constructions, and equivalents falling within spirit and scope of disclosure, as defined in appended claims.

Use of terms “a” and “an” and “the” and similar referents in context of describing disclosed embodiments (especially in context of following claims) are to be construed to cover both singular and plural, unless otherwise indicated herein or clearly contradicted by context, and not as a definition of a term. Terms “comprising,” “having,” “including,” and “containing” are to be construed as open-ended terms (meaning “including, but not limited to,”) unless otherwise noted. “Connected,” when unmodified and referring to physical connections, is to be construed as partly or wholly contained within, attached to, or joined together, even if there is something intervening. Recitation of ranges of values herein are merely intended to serve as a shorthand method of referring individually to each separate value falling within range, unless otherwise indicated herein and each separate value is incorporated into specification as if it were individually recited herein. In at least one embodiment, use of term “set” (e.g., “a set of items”) or “subset” unless otherwise noted or contradicted by context, is to be construed as a nonempty collection comprising one or more members. Further, unless otherwise noted or contradicted by context, term “subset” of a corresponding set does not necessarily denote a proper subset of corresponding set, but subset and corresponding set may be equal.

Conjunctive language, such as phrases of form “at least one of A, B, and C,” or “at least one of A, B and C,” unless specifically stated otherwise or otherwise clearly contradicted by context, is otherwise understood with context as used in general to present that an item, term, etc., may be either A or B or C, or any nonempty subset of set of A and B and C. For instance, in illustrative example of a set having three members, conjunctive phrases “at least one of A, B, and C” and “at least one of A, B and C” refer to any of following sets: {A}, {B}, {C}, {A, B}, {A, C}, {B, C}, {A, B, C}. Thus, such conjunctive language is not generally intended to imply that certain embodiments require at least one of A, at least one of B and at least one of C each to be present. In addition, unless otherwise noted or contradicted by context, term “plurality” indicates a state of being plural (e.g., “a plurality of items” indicates multiple items). In at least one embodiment, number of items in a plurality is at least two, but can be more when so indicated either explicitly or by context. Further, unless stated otherwise or otherwise clear from context, phrase “based on” means “based at least in part on” and not “based solely on.”

Operations of processes described herein can be performed in any suitable order unless otherwise indicated herein or otherwise clearly contradicted by context. In at least one embodiment, a process such as those processes described herein (or variations and/or combinations thereof) is performed under control of one or more computer systems configured with executable instructions and is implemented as code (e.g., executable instructions, one or more computer programs or one or more applications) executing collectively on one or more processors, by hardware or combinations thereof. In at least one embodiment, code is stored on a computer-readable storage medium, for example, in form of a computer program comprising a plurality of instructions executable by one or more processors. In at least one embodiment, a computer-readable storage medium is a non-transitory computer-readable storage medium that excludes transitory signals (e.g., a propagating transient electric or electromagnetic transmission) but includes non-transitory data storage circuitry (e.g., buffers, cache, and queues) within transceivers of transitory signals. In at least one embodiment, code (e.g., executable code or source code) is stored on a set of one or more non-transitory computer-readable storage media having stored thereon executable instructions (or other memory to store executable instructions) that, when executed (i.e., as a result of being executed) by one or more processors of a computer system, cause computer system to perform operations described herein. In at least one embodiment, set of non-transitory computer-readable storage media comprises multiple non-transitory computer-readable storage media and one or more of individual non-transitory storage media of multiple non-transitory computer-readable storage media lack all of code while multiple non-transitory computer-readable storage media collectively store all of code. In at least one embodiment, executable instructions are executed such that different instructions are executed by different processors —for example, a non-transitory computer-readable storage medium store instructions and a main central processing unit (“CPU”) executes some of instructions while a graphics processing unit (“GPU”) executes other instructions. In at least one embodiment, different components of a computer system have separate processors and different processors execute different subsets of instructions.

In at least one embodiment, an arithmetic logic unit is a set of combinational logic circuitry that takes one or more inputs to produce a result. In at least one embodiment, an arithmetic logic unit is used by a processor to implement mathematical operation such as addition, subtraction, or multiplication. In at least one embodiment, an arithmetic logic unit is used to implement logical operations such as logical AND/OR or XOR. In at least one embodiment, an arithmetic logic unit is stateless, and made from physical switching components such as semiconductor transistors arranged to form logical gates. In at least one embodiment, an arithmetic logic unit may operate internally as a stateful logic circuit with an associated clock. In at least one embodiment, an arithmetic logic unit may be constructed as an asynchronous logic circuit with an internal state not maintained in an associated register set. In at least one embodiment, an arithmetic logic unit is used by a processor to combine operands stored in one or more registers of the processor and produce an output that can be stored by the processor in another register or a memory location.

In at least one embodiment, as a result of processing an instruction retrieved by the processor, the processor presents one or more inputs or operands to an arithmetic logic unit, causing the arithmetic logic unit to produce a result based at least in part on an instruction code provided to inputs of the arithmetic logic unit. In at least one embodiment, the instruction codes provided by the processor to the ALU are based at least in part on the instruction executed by the processor. In at least one embodiment combinational logic in the ALU processes the inputs and produces an output which is placed on a bus within the processor. In at least one embodiment, the processor selects a destination register, memory location, output device, or output storage location on the output bus so that clocking the processor causes the results produced by the ALU to be sent to the desired location.

In the scope of this application, the term arithmetic logic unit, or ALU, is used to refer to any computational logic circuit that processes operands to produce a result. For example, in the present document, the term ALU can refer to a floating point unit, a DSP, a tensor core, a shader core, a coprocessor, or a CPU.

Accordingly, in at least one embodiment, computer systems are configured to implement one or more services that singly or collectively perform operations of processes described herein and such computer systems are configured with applicable hardware and/or software that enable performance of operations. Further, a computer system that implements at least one embodiment of present disclosure is a single device and, in another embodiment, is a distributed computer system comprising multiple devices that operate differently such that distributed computer system performs operations described herein and such that a single device does not perform all operations.

Use of any and all examples, or exemplary language (e.g., “such as”) provided herein, is intended merely to better illuminate embodiments of disclosure and does not pose a limitation on scope of disclosure unless otherwise claimed. No language in specification should be construed as indicating any non-claimed element as essential to practice of disclosure.

All references, including publications, patent applications, and patents, cited herein are hereby incorporated by reference to same extent as if each reference were individually and specifically indicated to be incorporated by reference and were set forth in its entirety herein.

In description and claims, terms “coupled” and “connected,” along with their derivatives, may be used. It should be understood that these terms may be not intended as synonyms for each other. Rather, in particular examples, “connected” or “coupled” may be used to indicate that two or more elements are in direct or indirect physical or electrical contact with each other. “Coupled” may also mean that two or more elements are not in direct contact with each other, but yet still co-operate or interact with each other.

Unless specifically stated otherwise, it may be appreciated that throughout specification terms such as “processing,” “computing,” “calculating,” “determining,” or like, refer to action and/or processes of a computer or computing system, or similar electronic computing device, that manipulate and/or transform data represented as physical, such as electronic, quantities within computing system's registers and/or memories into other data similarly represented as physical quantities within computing system's memories, registers or other such information storage, transmission or display devices.

In a similar manner, term “processor” may refer to any device or portion of a device that processes electronic data from registers and/or memory and transform that electronic data into other electronic data that may be stored in registers and/or memory. As non-limiting examples, “processor” may be a CPU or a GPU. A “computing platform” may comprise one or more processors. As used herein, “software” processes may include, for example, software and/or hardware entities that perform work over time, such as tasks, threads, and intelligent agents. Also, each process may refer to multiple processes, for carrying out instructions in sequence or in parallel, continuously or intermittently. In at least one embodiment, terms “system” and “method” are used herein interchangeably insofar as system may embody one or more methods and methods may be considered a system.

In present document, references may be made to obtaining, acquiring, receiving, or inputting analog or digital data into a subsystem, computer system, or computer-implemented machine. In at least one embodiment, process of obtaining, acquiring, receiving, or inputting analog and digital data can be accomplished in a variety of ways such as by receiving data as a parameter of a function call or a call to an application programming interface. In at least one embodiment, processes of obtaining, acquiring, receiving, or inputting analog or digital data can be accomplished by transferring data via a serial or parallel interface. In at least one embodiment, processes of obtaining, acquiring, receiving, or inputting analog or digital data can be accomplished by transferring data via a computer network from providing entity to acquiring entity. In at least one embodiment, references may also be made to providing, outputting, transmitting, sending, or presenting analog or digital data. In various examples, processes of providing, outputting, transmitting, sending, or presenting analog or digital data can be accomplished by transferring data as an input or output parameter of a function call, a parameter of an application programming interface or interprocess communication mechanism.

Although descriptions herein set forth example implementations of described techniques, other architectures may be used to implement described functionality, and are intended to be within scope of this disclosure. Furthermore, although specific distributions of responsibilities may be defined above for purposes of description, various functions and responsibilities might be distributed and divided in different ways, depending on circumstances.
1. In some embodiments, a method comprises inputting molecular data representative of at least a portion of a molecule generated during a first iteration of a molecular design process into one or more classifiers, wherein each classifier included in the one or more classifiers is trained using training data derived from at least one of one or more molecular dynamics simulations or one or more biological assays; generating, via execution of the one or more classifiers and based at least on the portion of the molecule, one or more scores, wherein each score included in the one or more scores represents a predicted measure of a different undesired attribute for at least the portion of the molecule; determining that the at least the portion of the molecule is associated with at least one undesired attribute based at least on a comparison of the one or more scores with one or more thresholds; and in response to the determination, preventing subsequent iterations of the molecular design process from using an input based at least on the portion of the molecule.
2. The method of clause 1, further comprising generating, via execution of the one or more classifiers and based at least on a portion of a second molecule, one or more additional scores; and outputting the second molecule as a drug candidate based at least on an additional comparison of the one or more additional scores with one or more additional thresholds.
3. The method of any of clauses 1-2, further comprising determining the one or more thresholds and the one or more additional thresholds based at least on a set of safety requirements associated with the molecule and the second molecule.
4. The method of claim any of clauses 1-3, further comprising generating, via execution of the one or more classifiers and based at least on a portion of a second molecule, one or more additional scores; and converting, based at least on the one or more additional scores, the second molecule into a third molecule during the molecular design process.
5. The method of any of clauses 1-4, wherein the training data is derived, at least, by performing a first set of molecular dynamics simulations associated with a set of molecules; and in response to determining that first set of simulation results associated with the first set of molecular dynamics simulations is inconclusive, performing a second set of molecular dynamics simulations, wherein the second set of molecular dynamics simulations is associated with a higher accuracy than the first set of molecular dynamics simulations.
6. The method of any of clauses 1-5, further comprising computing one or more losses based at least on the one or more scores; and updating one or more parameters of a generative model associated with the molecular design process based at least on the one or more losses.
7. The method of any of clauses 1-6, wherein the molecular data comprises at least one of a sequence of characters, a graph, an image, or a three-dimensional (3D) representation of at least the portion of the molecule.
8. The method of any of clauses 1-7, wherein the one or more classifiers comprise at least one of a tree-based model, a deep learning model, or an ensemble model.
9. The method of any of clauses 1-8, wherein the different undesired attribute is associated with at least one of a toxicity, an illegal substance, a protected substance, or binding to an off-target.
10. The method of any of clauses 1-9, wherein at least the portion of the molecule is generated using at least one of a diffusion model, a variational autoencoder, a normalizing flow model, or a generative adversarial network.
11. In some embodiments, at least one processor comprises processing circuitry to perform operations comprising inputting molecular data representative of at least a portion of a molecule generated during a first iteration of a molecular design process into one or more classifiers; generating, via execution of the one or more classifiers and based at least on the portion of the molecule, one or more scores respectively representing a predicted measure of a different undesired attribute for at least the portion of the molecule; determining that at least the portion of the molecule is associated with at least one undesired attribute based at least on a comparison of the one or more scores with one or more thresholds; and in response to the determination, preventing subsequent iterations of the molecular design process using an input based on the at least the portion of the molecule, wherein the preventing comprises filtering the molecular data from a second iteration of the molecular design process that follows the first iteration.
12. The at least one processor of clause 11, wherein the operations further comprise generating, via execution of the one or more classifiers and based at least on a portion of a second molecule, one or more additional scores; and outputting the second molecule or a third molecule derived from the second molecule as a drug candidate based at least on the one or more additional scores.
13. The at least one processor of any of clauses 11-12, wherein the operations further comprise generating, via execution of the one or more classifiers and based at least on a portion of a second molecule, one or more additional scores; and generating, based on the second molecule, a third molecule during one or more additional iterations of the molecular design process.
14. The at least one processor of any of clauses 11-13, wherein the operations further comprise generating, via execution of the one or more classifiers and based at least on a portion of a second molecule, one or more additional scores; and filtering the second molecule from a set of drug candidates based at least on an additional comparison of the one or more additional scores with one or more additional thresholds.
15. The at least one processor of any of clauses 11-14, wherein the operations further comprise generating, via execution of the one or more classifiers and based at least on input that includes at least a portion of a second molecule generated using a generative model, one or more additional scores; computing one or more losses based at least on the one or more additional scores; updating one or more parameters of the generative model based at least on the one or more losses to generate a trained generative model; and generating at least the portion of the molecule using the trained generative model.
16. The at least one processor of any of clauses 11-15, wherein the operations further comprise generating, via execution of the one or more classifiers and based at least on input that includes a latent representation of a second molecule generated using a trained generative model, one or more additional scores; and modifying generation of one or more additional latent representations of the second molecule using the trained generative model based at least on the one or more additional scores.
17. The at least one processor of any of clauses 11-16, wherein the predicted measure comprises at least one of a probability of toxicity, a level of toxicity, a similarity to an illegal substance, a similarity to a protected substance, a binding affinity to an off-target, or a likelihood of binding to an off-target.
18. The at least one processor of any of clauses 11-17, wherein the at least one processor is comprised in at least one of a system for performing simulation operations; a system for performing digital twin operations; a system for performing collaborative content creation for 3D assets; a system for performing one or more deep learning operations; a system implemented using an edge device; a system for generating or presenting at least one of virtual reality content, augmented reality content, or mixed reality content; a system implemented using a robot; a system for performing one or more conversational AI operations; a system implemented using one or more large language models (LLMs); a system implementing one or more vision language models (VLMs); a system implementing one or more multi modal language models; a system for generating synthetic data; a system for performing one or more generative AI operations; a system incorporating one or more virtual machines (VMs); a system implemented at least partially in a data center; or a system implemented at least partially using cloud computing resources.
19. In some embodiments, a system comprises one or more processors to perform operations comprising inputting molecular data representative of at least a portion of a molecule generated during a first iteration of a molecular design process into one or more classifiers; generating, based at least on the one or more classifiers processing the molecular data, one or more scores respectively representing a predicted measure of a different undesired attribute for at least the portion of the molecule; and based on at least one undesired attribute exceeding a threshold, at least one of filtering at least the portion of the molecule from a second iteration of the molecular design process; or preventing at least one of transmission or display of data corresponding to at least the portion of the molecule during the molecular design process.
20. The system of clause 19, wherein the system is comprised in at least one of wherein the at least one processor is comprised in at least one of a system for performing simulation operations; a system for performing digital twin operations; a system for performing collaborative content creation for 3D assets; a system for performing one or more deep learning operations; a system implemented using an edge device; a system for generating or presenting at least one of virtual reality content, augmented reality content, or mixed reality content; a system implemented using a robot; a system for performing one or more conversational AI operations; a system implemented using one or more large language models (LLMs); a system implementing one or more vision language models (VLMs); a system implementing one or more multi modal language models; a system for generating synthetic data; a system for performing one or more generative AI operations; a system incorporating one or more virtual machines (VMs); a system implemented at least partially in a data center; or a system implemented at least partially using cloud computing resources.
21. In some embodiments, a method comprises inputting a latent representation of a molecule generated using a trained generative model during a first generative instance into one or more classifiers, wherein each classifier included in the one or more classifiers is trained using training data derived from at least one of one or more molecular dynamics simulations or one or more biological assays; generating, via execution of the one or more classifiers and based at least on the latent representation of the molecule, one or more scores, wherein each score included in the one or more scores represents a predicted measure of a different undesired attribute for the latent representation of the molecule; determining that the one or more scores are not within one or more acceptable ranges; and in response to the determination, preventing the trained generative model from generating one or more second latent representations of the molecule, wherein the preventing comprises causing the trained generative model to generate, based at least on the one or more scores, one or more third latent representations associated with the molecule over one or more additional generative instances following the first generative instance.
22. The method of clause 21, further comprising generating, via execution of the one or more classifiers and based at least on a second latent representation of a second molecule generated using the trained generative model, one or more additional scores; and outputting the second molecule as a drug candidate based at least on a comparison of the one or more additional scores with one or more thresholds.
23. The method of any of clauses 21-22, further comprising generating, via execution of the one or more classifiers and based at least on a second latent representation of a second molecule generated using a generative model, one or more additional scores; computing one or more losses based on the one or more additional scores; and updating one or more parameters of the generative model based at least on the one or more losses to generate the trained generative model.
24. The method of any of clauses 21-13, wherein the training data is derived, at least, by performing a first set of molecular dynamics simulations associated with a set of molecules; and in response to determining that first set of simulation results associated with the first set of molecular dynamics simulations is inconclusive, performing a second set of molecular dynamics simulations, wherein the second set of molecular dynamics simulations is associated with a higher accuracy than the first set of molecular dynamics simulations.
25. The method of any of clauses 21-24, further comprising generating the latent representation of the molecule, at least, by sampling from a latent space associated with the trained generative model.
26. The method of any of clauses 21-25, further comprising generating the latent representation of the molecule via a denoising process associated with the trained generative model.
27. The method of any of clauses 21-26, wherein the causing the trained generative model to generate the one or more third latent representations of the molecule comprises at least one of sampling the one or more third latent representations of the molecule based at least on the one or more scores; converting the latent representation of the molecule into the one or more third latent representations based at least on the one or more scores; or conditioning generation of the one or more third latent representations of the molecule by the trained generative model based at least on the one or more scores.
28. The method of any of clauses 21-27, wherein the one or more classifiers comprise at least one of a tree-based model, a deep learning model, an ensemble model, a transformer neural network, a feedforward neural network, or a graph neural network.
29. The method of any of clauses 21-28, wherein the trained generative model comprises at least one of a diffusion model, a variational autoencoder, a normalizing flow model, or a generative adversarial network.
30. The method of any of clauses 21-29, wherein the different undesired attribute is associated with at least one of a toxicity, an illegal substance, a protected substance, or binding to an off-target.
31. In some embodiments, at least one processor comprises processing circuitry to perform operations comprising inputting a latent representation of a molecule generated by a trained generative model during a first generative timestep into one or more classifiers; generating, via execution of the one or more classifiers and based on at least the latent representation of the molecule, one or more scores, wherein each score included in the one or more scores represents a predicted measure of a different undesired attribute for the latent representation of the molecule; determining that the one or more scores are not within one or more acceptable ranges; and in response to the determination, preventing the trained generative model from generating one or more additional latent representations of the molecule, wherein the preventing comprises modifying, based at least on the one or more scores, generation associated with the one or more additional latent representations of the molecule by the trained generative model over one or more additional generative timesteps following the first generative timestep.
32. The at least one processor of claim 31, wherein the operations further comprise generating, via execution of the one or more classifiers based at least on a second latent representation of a second molecule generated by the trained generative model, one or more additional scores; and outputting the second molecule as a drug candidate based on a comparison of the one or more additional scores with one or more thresholds.
33. The at least one processor of any of clauses 31-32, wherein the second latent representation is generated by the trained generative model based on at least one of a prompt, a sequence of characters, a graph, an image, an additional latent representation, or a three-dimensional (3D) representation.
34. The at least one processor of any of clauses 31-33, wherein the operations further comprise generating, via execution of the one or more classifiers based at least on a second latent representation of a second molecule generated by a generative model, one or more additional scores; computing one or more losses based on the one or more additional scores; and training the generative model based at least on the one or more losses to generate the trained generative model.
35. The at least one processor of any of clauses 31-34, wherein the one or more classifiers comprise at least one of a tree-based model, a deep learning model, an ensemble model, a transformer neural network, a feedforward neural network, or a graph neural network.
36. The at least one processor of any of clauses 31-35, wherein the latent representation of the molecule is generated using at least one of a sample from a latent space associated with the trained generative model; or a denoising process associated with the trained generative model.
37. The at least one processor of any of clauses 31-36, wherein the predicted measure of the different undesired attribute comprises at least one of a probability of toxicity, a level of toxicity, a similarity to an illegal substance, a similarity to a protected substance, a binding affinity to an off-target, or a likelihood of binding to an off-target.
38. The at least one processor of any of clauses 31-37, wherein the at least one processor is comprised in at least one of a system for performing simulation operations; a system for performing digital twin operations; a system for performing collaborative content creation for 3D assets; a system for performing one or more deep learning operations; a system implemented using an edge device; a system for generating or presenting at least one of virtual reality content, augmented reality content, or mixed reality content; a system implemented using a robot; a system for performing one or more conversational AI operations; a system implemented using one or more large language models (LLMs); a system implementing one or more vision language models (VLMs); a system implementing one or more multi modal language models; a system for generating synthetic data; a system for performing one or more generative AI operations; a system incorporating one or more virtual machines (VMs); a system implemented at least partially in a data center; or a system implemented at least partially using cloud computing resources.
39. In some embodiments, a system comprises one or more processors to perform operations comprising inputting a latent representation of a molecule generated using a trained generative model into one or more classifiers; generating, based at least on the one or more classifiers processing at least the latent representation of the molecule, one or more scores respectively representing a predicted measure of a different undesired attribute for the latent representation of the molecule; determining that the one or more scores are not within one or more acceptable ranges; and in response to the determination, preventing the trained generative model from generating one or more additional latent representations of the molecule.
40. The system of clause 39, wherein the system is comprised in at least one of a system for performing simulation operations; a system for performing digital twin operations; a system for performing collaborative content creation for 3D assets; a system for performing one or more deep learning operations; a system implemented using an edge device; a system for generating or presenting at least one of virtual reality content, augmented reality content, or mixed reality content; a system implemented using a robot; a system for performing one or more conversational AI operations; a system implemented using one or more large language models (LLMs); a system implementing one or more vision language models (VLMs); a system implementing one or more multi modal language models; a system for generating synthetic data; a system for performing one or more generative AI operations; a system incorporating one or more virtual machines (VMs); a system implemented at least partially in a data center; or a system implemented at least partially using cloud computing resources.
41. In some embodiments, a method comprises generating, using a generative model, molecular data representative of at least a portion of a molecule; inputting the molecular data into one or more classifiers respectively trained using training data derived from at least one of one or more molecular dynamics simulations or one or more biological assays; generating, via execution of the one or more classifiers and based at least on the molecular data, one or more scores, wherein each score included in the one or more scores represents a predicted measure of a different undesired attribute for the at least the portion of the molecule; computing one or more losses corresponding to the generative model based at least on the one or more scores; and updating one or more parameters of the generative model based at least on the one or more losses to generate a trained generative model.
42. The method of clause 41, further comprising generating at least a portion of a second molecule using the trained generative model.
43. The method of any of clauses 41-42, further comprising generating, via execution of the one or more classifiers and based at least on the portion of the second molecule, one or more additional scores; and outputting the second molecule or a third molecule derived from the second molecule as a drug candidate based at least on the one or more additional scores.
44. The method of any of clauses 41-43, further comprising: generating, via execution of the one or more classifiers and based at least on a latent representation of a second molecule generated using the generative model, one or more additional scores; computing one or more additional losses based at least on the one or more additional scores; and updating the one or more parameters of the generative model based at least on the one or more additional losses.
45. The method of any of clauses 41-44, further comprising computing one or more additional losses based on at least one of (i) at least the portion of the molecule or (ii) a latent representation of at least the portion of the molecule generated by the generative model; and updating the one or more parameters of the generative model based at least on the one or more additional losses.
46. The method of any of clauses 41-45, wherein at least the portion of the molecule comprises at least one of a sequence of characters, a graph, an image, or a three-dimensional (3D) representation.
47. The method of any of clauses 41-46, wherein the one or more classifiers comprise at least one of a tree-based model, a deep learning model, or an ensemble model.
48. The method of any of clauses 41-47, wherein the different undesired attribute is associated with at least one of a toxicity, an illegal substance, a protected substance, or binding to an off-target.
49. The method of any of clauses 41-48, wherein the generative model comprises at least one of a diffusion model, a variational autoencoder, a normalizing flow model, or a generative adversarial network.
50. The method of any of clauses 41-48, wherein the one or more losses comprise at least one of an adversarial loss, a Kullback-Leibler divergence, a parameterization of a distribution associated with the generative model, a reconstruction loss, a mean squared error, a cross-entropy loss, or a perceptual loss.
51. In some embodiments, at least one processor comprises processing circuitry to perform operations comprising inputting molecular data representative of one or more representations of at least a portion of a molecule generated by a generative model into one or more classifiers, wherein each classifier included in the one or more classifiers is trained using training data derived from at least one of one or more molecular dynamics simulations or one or more biological assays; generating, via execution of the one or more classifiers based at least on at least the portion of the molecule, one or more scores, wherein each score included in the one or more scores represents a predicted measure of a different undesired attribute for the at least the portion of the molecule; computing one or more losses of the generative model based on the one or more scores; and training the generative model based at least on the one or more losses to generate a trained generative model.
52. The at least one processor of clause 51, wherein the operations further comprise generating at least a portion of a second molecule using the trained generative model.
53. The at least one processor of any of clauses 51-52, wherein the operations further comprise generating, via execution of the one or more classifiers based at least on the portion of the second molecule, one or more additional scores; and filtering at least the portion of the second molecule based on a comparison of the one or more additional scores with one or more thresholds.
54. The at least one processor of any of clauses 51-53, wherein the operations further comprise generating, via execution of the one or more classifiers based at least on a latent representation of a second molecule generated by the trained generative model, one or more additional scores; and modifying, based at least on the one or more additional scores, generation of one or more additional latent representations of the second molecule by the trained generative model.
55. The at least one processor of any of clauses 51-54, wherein the training data is derived by performing a first set of molecular dynamics simulations associated with a set of molecules; and in response to determining that first set of simulation results associated with the first set of molecular dynamics simulations is inconclusive, performing a second set of molecular dynamics simulations, wherein the second set of molecular dynamics simulations is associated with a higher accuracy than the first set of molecular dynamics simulations.
56. The at least one processor of any of clauses 51-55, wherein the one or more losses comprise at least one of an adversarial loss, a Kullback-Leibler divergence, a parameterization of a distribution associated with the generative model, a reconstruction loss, a mean squared error, a cross-entropy loss, or a perceptual loss.
57. The at least one processor of any of clauses 51-56, wherein the one or more representations comprise at least one of a sequence of characters, a graph, an image, a three-dimensional (3D) representation, or a latent representation.
58. The at least one processor of any of clauses 51-57, wherein the at least one processor is comprised in at least one of a system for performing simulation operations; a system for performing digital twin operations; a system for performing collaborative content creation for 3D assets; a system for performing one or more deep learning operations; a system implemented using an edge device; a system for generating or presenting at least one of virtual reality content, augmented reality content, or mixed reality content; a system implemented using a robot; a system for performing one or more conversational AI operations; a system implemented using one or more large language models (LLMs); a system implementing one or more vision language models (VLMs); a system implementing one or more multi modal language models; a system for generating synthetic data; a system for performing one or more generative AI operations; a system incorporating one or more virtual machines (VMs); a system implemented at least partially in a data center; or a system implemented at least partially using cloud computing resources.
59. In some embodiments, a system comprises one or more processors to perform operations comprising generating, using a generative model, molecular data representative of a least a portion of a molecule; generating, based at least on one or more classifiers processing the molecular data, one or more scores respectively representing a predicted measure of a different undesired attribute for at least the portion of the molecule; computing one or more losses corresponding to the generative model based at least on the one or more scores; and updating one or more parameters of the generative model based at least on the one or more losses to generate a trained generative model.
60. The system of clause 59, wherein the system is comprised in at least one of a system for performing simulation operations; a system for performing digital twin operations; a system for performing collaborative content creation for 3D assets; a system for performing one or more deep learning operations; a system implemented using an edge device; a system for generating or presenting at least one of virtual reality content, augmented reality content, or mixed reality content; a system implemented using a robot; a system for performing one or more conversational AI operations; a system implemented using one or more large language models (LLMs); a system implementing one or more vision language models (VLMs); a system implementing one or more multi modal language models; a system for generating synthetic data; a system for performing one or more generative AI operations; a system incorporating one or more virtual machines (VMs); a system implemented at least partially in a data center; or a system implemented at least partially using cloud computing resources.
61. In some embodiments, a method comprises inputting a representation of at least a portion of a prompt associated with a trained generative model into one or more classifiers; generating, via execution of the one or more classifiers and based at least on the representation, one or more scores, wherein each score included in the one or more scores represents a predicted measure of a different undesired attribute associated with a molecule to be generated using the trained generative model based on the prompt; determining that the at least the portion of the prompt is associated with at least one undesired attribute based at least on a comparison of the one or more scores with one or more thresholds; and in response to the determination, preventing the prompt from being applied to the trained generative model, wherein the preventing comprises filtering the prompt as input into the trained generative model.
62. The method of clause 61, further comprising converting at least the portion of the prompt into the representation in a latent space.
63. The method of any of clauses 61-62, wherein at least one score included in the one or more scores is generated based at least on a distance between the representation and an additional representation of a restricted molecule in the latent space.
64. The method of any of clauses 61-63, further comprising generating, via execution of the one or more classifiers and based at least on a portion of a second prompt associated with the trained generative model, one or more additional scores; and causing the second prompt to be inputted into the trained generative model based at least on a comparison of the one or more additional scores with the one or more thresholds.
65. The method of any of clauses 61-64, further comprising generating, via execution of the one or more classifiers and based at least on a latent representation of a second molecule generated using a generative model, one or more additional scores; computing one or more losses based on the one or more additional scores; and updating one or more parameters of the generative model based at least on the one or more losses to generate the trained generative model.
66. The method of any of clauses 61-65, wherein the one or more classifiers comprise at least one of a set of rules, one or more filters, a named entity recognition technique, a search technique, or a symbolic model.
67. The method of any of clauses 61-66, wherein the one or more classifiers comprise at least one of a tree-based model, a deep learning model, or an ensemble model.
68. The method of any of clauses 61-67, wherein the different undesired attribute is associated with at least one of a toxicity, an illegal substance, a protected substance, or binding to an off-target.
69. The method of any of clauses 61-68, wherein the trained generative model comprises at least one of a multimodal language model, a diffusion model, a variational encoder, a generative adversarial network, or a large language model.
70. The method of any of clauses 61-69, wherein the at least the portion of the prompt comprises at least one of an instruction to generate the molecule or a representation of the molecule.
71. In some embodiments, at least one processor comprises processing circuitry to perform operations comprising inputting a representation of at least a portion of a prompt associated with a trained generative model into one or more classifiers; generating, via execution of the one or more classifiers and based at least on the representation, one or more scores, wherein each score included in the one or more scores represents a predicted measure of a different undesired attribute associated with a molecule to be generated using the trained generative model based on the prompt; determining that the at least the portion of the prompt is associated with at least one undesired attribute based on a comparison of the one or more scores with one or more thresholds; and in response to the determination, preventing the prompt from being applied to the trained generative model, wherein the preventing comprises filtering the prompt as input into the trained generative model.
72. The at least one processor of clause 71, wherein the operations further comprise populating the representation with one or more attributes extracted from the prompt.
73. The at least one processor of any of clauses 71-72, wherein the operations further comprise generating, via execution of the one or more classifiers and based at least on at least a portion of a second molecule generated by the trained generative model, one or more additional scores; and filtering at least a portion of the second molecule based on a comparison of the one or more additional scores with one or more additional thresholds.
74. The at least one processor of claim any of clauses 71-73, wherein the operations further comprise generating, via execution of the one or more classifiers based at least on a latent representation of a second molecule generated by the trained generative model, one or more additional scores; and modifying, based at least on the one or more additional scores, generation of one or more additional latent representations of the second molecule by the trained generative model.
75. The at least one processor of any of clauses 71-74, wherein the operations further comprise generating, via execution of the one or more classifiers based at least on at least a portion of a second prompt associated with the trained generative model, one or more additional scores; and modifying, based on a comparison of the one or more additional scores with the one or more thresholds, generation of a second molecule by the trained generative model using the second prompt.
76. The at least one processor of any of clauses 71-75, wherein the at least the portion of the prompt comprises at least one of an instruction to generate the molecule, an instruction to generate a synthesis route for the molecule, or a representation of the molecule.
77. The at least one processor of any of clauses 71-76, wherein the one or more classifiers comprise at least one of a machine learning model or a symbolic model.
78. The at least one processor of any of clauses 71-77, wherein the at least one processor is comprised in at least one of a system for performing simulation operations; a system for performing digital twin operations; a system for performing collaborative content creation for 3D assets; a system for performing one or more deep learning operations; a system implemented using an edge device; a system for generating or presenting at least one of virtual reality content, augmented reality content, or mixed reality content; a system implemented using a robot; a system for performing one or more conversational AI operations; a system implemented using one or more large language models (LLMs); a system implementing one or more vision language models (VLMs); a system implementing one or more multi modal language models; a system for generating synthetic data; a system for performing one or more generative AI operations; a system incorporating one or more virtual machines (VMs); a system implemented at least partially in a data center; or a system implemented at least partially using cloud computing resources.
79. In some embodiments, a system comprises one or more processors to perform operations comprising inputting a representation of at least a portion of a prompt associated with a trained generative model into one or more classifiers; generating, based at least on the one or more classifiers processing the representation, one or more scores respectively representing a predicted measure of a different undesired attribute associated with a molecule to be generated, based on the prompt, using the trained generative model; and based on the one or more scores, at least one of preventing the prompt from being applied to the trained generative model; or preventing transmission or presentation of an output of the trained generative model generated using the prompt.
80. The system of clause 79, wherein the system is comprised in at least one of a system for performing simulation operations; a system for performing digital twin operations; a system for performing collaborative content creation for 3D assets; a system for performing one or more deep learning operations; a system implemented using an edge device; a system for generating or presenting at least one of virtual reality content, augmented reality content, or mixed reality content; a system implemented using a robot; a system for performing one or more conversational AI operations; a system implemented using one or more large language models (LLMs); a system implementing one or more vision language models (VLMs); a system implementing one or more multi modal language models; a system for generating synthetic data; a system for performing one or more generative AI operations; a system incorporating one or more virtual machines (VMs); a system implemented at least partially in a data center; or a system implemented at least partially using cloud computing resources.

Furthermore, although subject matter has been described in language specific to structural features and/or methodological acts, it is to be understood that subject matter claimed in appended claims is not necessarily limited to specific features or acts described. Rather, specific features and acts are disclosed as exemplary forms of implementing the claims.

What is claimed is:

1. A method comprising:
inputting a latent representation of a molecule generated using a trained generative model during a first generative instance into one or more classifiers, wherein each classifier included in the one or more classifiers is trained using training data derived from at least one of one or more molecular dynamics simulations or one or more biological assays;
generating, via execution of the one or more classifiers and based at least on the latent representation of the molecule, one or more scores, wherein each score included in the one or more scores represents a predicted measure of a different undesired attribute for the latent representation of the molecule;
determining that the one or more scores are not within one or more acceptable ranges; and
in response to the determination, preventing the trained generative model from generating one or more second latent representations of the molecule, wherein the preventing comprises causing the trained generative model to generate, based at least on the one or more scores, one or more third latent representations associated with the molecule over one or more additional generative instances following the first generative instance.

2. The method of claim 1, further comprising:
generating, via execution of the one or more classifiers and based at least on a second latent representation of a second molecule generated using the trained generative model, one or more additional scores; and
outputting the second molecule as a drug candidate based at least on a comparison of the one or more additional scores with one or more thresholds.

3. The method of claim 1, further comprising:
generating, via execution of the one or more classifiers and based at least on a second latent representation of a second molecule generated using a generative model, one or more additional scores;
computing one or more losses based on the one or more additional scores; and
updating one or more parameters of the generative model based at least on the one or more losses to generate the trained generative model.

4. The method of claim 1, wherein the training data is derived, at least, by:
performing a first set of molecular dynamics simulations associated with a set of molecules; and
in response to determining that first set of simulation results associated with the first set of molecular dynamics simulations is inconclusive, performing a second set of molecular dynamics simulations, wherein the second set of molecular dynamics simulations is associated with a higher accuracy than the first set of molecular dynamics simulations.

5. The method of claim 1, further comprising generating the latent representation of the molecule, at least, by sampling from a latent space associated with the trained generative model.

6. The method of claim 1, further comprising generating the latent representation of the molecule via a denoising process associated with the trained generative model.

7. The method of claim 1, wherein the causing the trained generative model to generate the one or more third latent representations of the molecule comprises at least one of:
sampling the one or more third latent representations of the molecule based at least on the one or more scores;
converting the latent representation of the molecule into the one or more third latent representations based at least on the one or more scores; or
conditioning generation of the one or more third latent representations of the molecule by the trained generative model based at least on the one or more scores.

8. The method of claim 1, wherein the one or more classifiers comprise at least one of a tree-based model, a deep learning model, an ensemble model, a transformer neural network, a feedforward neural network, or a graph neural network.

9. The method of claim 1, wherein the trained generative model comprises at least one of a diffusion model, a variational autoencoder, a normalizing flow model, or a generative adversarial network.

10. The method of claim 1, wherein the different undesired attribute is associated with at least one of a toxicity, an illegal substance, a protected substance, or binding to an off-target.

11. At least one processor comprising:
processing circuitry to perform operations comprising:
inputting a latent representation of a molecule generated by a trained generative model during a first generative timestep into one or more classifiers;
generating, via execution of the one or more classifiers and based on at least the latent representation of the molecule, one or more scores, wherein each score included in the one or more scores represents a predicted measure of a different undesired attribute for the latent representation of the molecule;
determining that the one or more scores are not within one or more acceptable ranges; and
in response to the determination, preventing the trained generative model from generating one or more additional latent representations of the molecule, wherein the preventing comprises modifying, based at least on the one or more scores, generation associated with the one or more additional latent representations of the molecule by the trained generative model over one or more additional generative timesteps following the first generative timestep.

12. The at least one processor of claim 11, wherein the operations further comprise:
generating, via execution of the one or more classifiers based at least on a second latent representation of a second molecule generated by the trained generative model, one or more additional scores; and
outputting the second molecule as a drug candidate based on a comparison of the one or more additional scores with one or more thresholds.

13. The at least one processor of claim 12, wherein the second latent representation is generated by the trained generative model based on at least one of a prompt, a sequence of characters, a graph, an image, an additional latent representation, or a three-dimensional (3D) representation.

14. The at least one processor of claim 11, wherein the operations further comprise:
generating, via execution of the one or more classifiers based at least on a second latent representation of a second molecule generated by a generative model, one or more additional scores;
computing one or more losses based on the one or more additional scores; and
training the generative model based at least on the one or more losses to generate the trained generative model.

15. The at least one processor of claim 11, wherein the one or more classifiers comprise at least one of a tree-based model, a deep learning model, an ensemble model, a transformer neural network, a feedforward neural network, or a graph neural network.

16. The at least one processor of claim 11, wherein the latent representation of the molecule is generated using at least one of:
a sample from a latent space associated with the trained generative model; or
a denoising process associated with the trained generative model.

17. The at least one processor of claim 11, wherein the predicted measure of the different undesired attribute comprises at least one of a probability of toxicity, a level of toxicity, a similarity to an illegal substance, a similarity to a protected substance, a binding affinity to an off-target, or a likelihood of binding to an off-target.

18. The at least one processor of claim 11, wherein the at least one processor is comprised in at least one of:
a system for performing simulation operations;
a system for performing digital twin operations;
a system for performing collaborative content creation for 3D assets;
a system for performing one or more deep learning operations;
a system implemented using an edge device;
a system for generating or presenting at least one of virtual reality content, augmented reality content, or mixed reality content;
a system implemented using a robot;
a system for performing one or more conversational AI operations;
a system implemented using one or more large language models (LLMs);
a system implementing one or more vision language models (VLMs);
a system implementing one or more multi modal language models;
a system for generating synthetic data;
a system for performing one or more generative AI operations;
a system incorporating one or more virtual machines (VMs);
a system implemented at least partially in a data center; or
a system implemented at least partially using cloud computing resources.

19. A system comprising:
one or more processors to perform operations comprising:
inputting a latent representation of a molecule generated using a trained generative model into one or more classifiers;
generating, based at least on the one or more classifiers processing at least the latent representation of the molecule, one or more scores respectively representing a predicted measure of a different undesired attribute for the latent representation of the molecule;
determining that the one or more scores are not within one or more acceptable ranges; and
in response to the determination, preventing the trained generative model from generating one or more additional latent representations of the molecule.

20. The system of claim 19, wherein the system is comprised in at least one of:
a system for performing simulation operations;
a system for performing digital twin operations;
a system for performing collaborative content creation for 3D assets;
a system for performing one or more deep learning operations;
a system implemented using an edge device;
a system for generating or presenting at least one of virtual reality content, augmented reality content, or mixed reality content;
a system implemented using a robot;
a system for performing one or more conversational AI operations;
a system implemented using one or more large language models (LLMs);
a system implementing one or more vision language models (VLMs);
a system implementing one or more multi modal language models;
a system for generating synthetic data;
a system for performing one or more generative AI operations;
a system incorporating one or more virtual machines (VMs);
a system implemented at least partially in a data center; or
a system implemented at least partially using cloud computing resources.
